(19) United States
(12) Patent Application Publication
(10) Pub. No.: US 2024/0338722 A1
(43) Pub. Date: Oct. 10, 2024

(54) INFORMATION PROCESSING DEVICE, INFORMATION PROCESSING SYSTEM, INFORMATION PROCESSING METHOD, AND PROGRAM

(71) Applicant: Sony Group Corporation, Tokyo (JP)

(72) Inventors: Kiminobu NISHIMURA, Kanagawa (JP); QiHong WANG, Tokyo (JP); Yasuhiro HORI, Kanagawa (JP)

(73) Assignee: Sony Group Corporation, Tokyo (JP)

(21) Appl. No.: 18/578,176

(22) PCT Filed: Mar. 2, 2022
(86) PCT No.: PCT/JP2022/008880

§ 371 Date: Jan. 10, 2024

(30) Foreign Application Priority Data
Jul. 21, 2021 (JP) 2021-120537

(51) Int. Cl. G06Q 30/0207; G06F 3/01; G06Q 40/08

(52) U.S. Cl. CPC G06Q 30/0207; G06F 3/011; G06Q 40/08

(57) ABSTRACT

Provided is an information processing device including a sensing data acquisition unit (246) that acquires a plurality of pieces of sensing data from a device worn by a user or carried by the user, a calculation unit (248) that calculates the number of steps or a movement distance of the user based on inertial data included in the plurality of pieces of sensing data, a reliability calculation unit (252) that calculates reliability based on a feature of each of position data, biological data, and environment data about the user obtained from the plurality of pieces of sensing data, a determination unit (254) that determines whether to accept the number of steps or movement distance calculated, based on the reliability calculated, and an output unit (256) that outputs data of the number of steps or movement distance received.

### FIELD

The present disclosure relates to an information processing device, an information processing system, an information processing method, and a program.

### BACKGROUND

In recent years, with increasing health consciousness, various services for health promotion have been proposed. One of such services is an insurance product called health promotion medical insurance. In life insurance and medical insurance, an insurance premium is determined on the basis of attribute information (age, sex, address, occupation, medical history, smoking history, etc.) of an insured person, but in the health promotion insurance, the health condition and health enhancement efforts (e.g., walking) of the insured person are evaluated, and a discount of insurance premium or repayment thereof is given according to the evaluation. According to such insurance, the insured person actively and continuously promotes health enhancement, such as walking, even after purchasing an insurance policy, in order to obtain an incentive such as the discount of insurance premium or repayment thereof.

### CITATION LIST

### Patent Literature

Patent Literature 1: JP 2018-23768 A

### SUMMARY

### Technical Problem

However, the creation of such a product as described above makes some insured persons falsify the number of steps or movement distance, such as measurement of the number of steps or movement distance with pedometers or the like by an improper method, instead of measurement of the number of steps or movement distance in actual walking, in order to obtain fraudulent incentives.

Therefore, the present disclosure proposes an information processing device, an information processing system, an information processing method, and a program that are configured to prevent falsification of the number of steps or the like.

### Solution to Problem

According to the present disclosure, there is provided an information processing device including: a sensing data acquisition unit that acquires a plurality of pieces of sensing data from a device worn by a user or carried by the user; a calculation unit that calculates the number of steps or a movement distance of the user based on inertial data included in the plurality of pieces of sensing data; a reliability calculation unit that calculates reliability based on a feature of each of position data, biological data, and environment data about the user obtained from the plurality of pieces of sensing data; a determination unit that determines whether to accept the number of steps or movement distance calculated, based on the reliability calculated; and an output unit that outputs data of the number of steps or movement distance received.

Furthermore, according to the present disclosure, there is provided an information processing system including: a server that calculates an incentive to a user; and an information processing device that is worn by the user or carried by the user. In the information processing system, the information processing device includes: a sensing data acquisition unit that acquires a plurality of pieces of sensing data from a device worn by the user or carried by the user; a calculation unit that calculates the number of steps or a movement distance of the user based on inertial data included in the plurality of pieces of sensing data; a reliability calculation unit that calculates reliability based on a feature of each of position data, biological data, and environment data about the user obtained from the plurality of pieces of sensing data; a determination unit that determines whether to accept the number of steps or movement distance calculated, based on the reliability calculated; an output unit that outputs, to the server, data of the number of steps or movement distance received; and a presentation unit that presents, to the user, the incentive calculated by the server based on the data of the number of steps or movement distance.

Furthermore, according to the present disclosure, there is provided an information processing method including: acquiring a plurality of pieces of sensing data from a device worn by a user or carried by the user; calculating the number of steps or a movement distance of the user based on inertial data included in the plurality of pieces of sensing data; calculating reliability based on a feature of each of position data, biological data, and environment data about the user obtained from the plurality of pieces of sensing data; determining whether to accept the number of steps or movement distance calculated, based on the reliability calculated; and outputting data of the number of steps or movement distance received, by an information processing device.

Furthermore, according to the present disclosure, there is provided a program causing a computer to perform: a function of acquiring a plurality of pieces of sensing data from a device worn by a user or carried by the user; a function of calculating the number of steps or a movement distance of the user based on inertial data included in the plurality of pieces of sensing data; a function of calculating reliability based on a feature of each of position data, biological data, and environment data about the user obtained from the plurality of pieces of sensing data; a function of determining whether to accept the number of steps or movement distance calculated, based on the reliability calculated; and a function of outputting data of the number of steps or movement distance received.

### BRIEF DESCRIPTION OF DRAWINGS

FIG. 1 is an illustrative diagram of an exemplary configuration of an information processing system 10 according to an embodiment of the present disclosure.

FIG. 2 is an illustrative diagram illustrating an exemplary appearance of a wearable device 100 according to an embodiment of the present disclosure.

FIG. 3 is a block diagram illustrating an exemplary configuration of the wearable device 100 according to an embodiment of the present disclosure.

FIG. 4 is a block diagram illustrating an exemplary configuration of a mobile device 200 according to an embodiment of the present disclosure.

FIG. 5 is a block diagram illustrating an exemplary configuration of a server 300 according to an embodiment of the present disclosure.

FIG. 6 is a sequence diagram illustrating an exemplary information processing method according to an embodiment of the present disclosure.

FIG. 7 is an illustrative diagram (1) illustrating an exemplary display screen according to an embodiment of the present disclosure.

FIG. 8 is an illustrative diagram (2) illustrating an exemplary display screen according to an embodiment of the present disclosure.

FIG. 9 is an illustrative diagram (3) illustrating an exemplary display screen according to an embodiment of the present disclosure.

FIG. 10 is an illustrative diagram (4) illustrating an exemplary display screen according to an embodiment of the present disclosure.

FIG. 11 is an illustrative diagram (5) illustrating an exemplary display screen according to an embodiment of the present disclosure.

FIG. 12 is an illustrative diagram (6) illustrating an exemplary display screen according to an embodiment of the present disclosure.

FIG. 13 is an illustrative diagram (7) illustrating an exemplary display screen according to an embodiment of the present disclosure.

FIG. 14 is an illustrative diagram (8) illustrating an exemplary display screen according to an embodiment of the present disclosure.

FIG. 15 is an illustrative diagram (9) illustrating an exemplary display screen according to an embodiment of the present disclosure.

FIG. 16 is a flowchart illustrating an exemplary information processing method according to an embodiment of the present disclosure.

FIG. 17 is an illustrative diagram (1) of an example of a feature in an embodiment of the present disclosure.

FIG. 18 is an illustrative diagram (2) of an example of a feature in an embodiment of the present disclosure.

FIG. 19 is an illustrative diagram (3) of an example of a feature in an embodiment of the present disclosure.

FIG. 20A is an illustrative diagram (4) of an example of a feature in an embodiment of the present disclosure.

FIG. 20B is an illustrative diagram (5) of an example of a feature in an embodiment of the present disclosure.

FIG. 21 is an illustrative diagram (6) of an example of a feature in an embodiment of the present disclosure.

FIG. 22 is an illustrative diagram illustrating reliability in an embodiment of the present disclosure.

FIG. 23 is a table (1) illustrating exemplary coefficients in an embodiment of the present disclosure.

FIG. 24 is a table (2) illustrating exemplary coefficients in an embodiment of the present disclosure.

FIG. 25 is a block diagram illustrating an example of a schematic functional configuration of a smartphone.

### DESCRIPTION OF EMBODIMENTS

Preferred embodiments of the present disclosure will be described in detail below with reference to the accompanying drawings. Note that in the present description and the drawings, component elements having substantially the same functional configurations are denoted by the same reference numerals, and redundant descriptions thereof will be omitted. Furthermore, in the present description and the drawings, a plurality of component elements having substantially the same functional configurations is distinguished by giving the same reference numerals followed by different alphabets in some cases. However, when there is no need to particularly distinguish the plurality of component elements having substantially the same or similar functional configuration, the components are denoted by the same reference numeral alone.

Note that the description will be given in the following order.
1. Background to create embodiments of present disclosure
2. Embodiments
2.1 Overview of information processing system 10 according to embodiment of present disclosure
2.2 Detailed configuration of wearable device 100
2.3 Detailed configuration of mobile device 200
2.4 Detailed configuration of server 300
2.5 Information processing method
2.6 Calculation of number of steps
2.7 Features
2.8 Calculation of reliability
3. Conclusion
4. Hardware configuration
5. Supplementary notes

### 1. BACKGROUND TO CREATE EMBODIMENTS OF PRESENT DISCLOSURE

First, before description of the embodiments of the present disclosure, the background to create the embodiments of the present disclosure by the present inventors will be briefly described.

As described above, with increasing health consciousness, various services for health promotion have been devised. One of such services is an insurance product called health promotion medical insurance. In life insurance and medical insurance, an insurance premium is determined on the basis of attribute information (age, sex, address, occupation, medical history, smoking history, etc.) of an insured person, but in the health promotion insurance, the health condition and health enhancement efforts of the insured person are evaluated, and a discount of insurance premium or repayment thereof is given according to the evaluation. According to such insurance, the insured person actively and continuously promotes health enhancement, even after purchasing an insurance policy, in order to obtain an incentive such as the discount of insurance premium or repayment thereof. For example, daily walking is one of the efforts for health promotion for which the incentive can be obtained. Specifically, the number of steps of the insured person is detected, and in a case where the number of steps is expected to be enough to obtain the effect of promoting the health of the insured person, an insurance company provides the above-described incentive depending on the number of steps, to the insured person.

However, the creation of such a product as described above makes some insured persons falsify the number of steps, in order to obtain fraudulent incentives. For example, the insured persons who perform such a fraudulent act arbitrarily give vibrations to devices that measure the number of steps to cause the devices to count the number of steps even though the persons are not actually walking. Specifically, the number of steps is detected by calculated through analysis of the acceleration data, and therefore, it is difficult to distinguish a change in acceleration caused by a vibrator from a change in acceleration caused by actual walking. In addition, it is conceivable to detect the number of steps by using a global navigation satellite system (GNSS) signal, but it is difficult to accurately detect the GNSS signal indoors, with inevitable measurement error increased. Furthermore, calculation of the insurance premium (discount amount) with the number of steps including a large error gives a sense of dissatisfaction with the insurance premium to an insured person who does not perform the fraudulent act as described above, leading to the reduction in the number of insurance subscribers.

Therefore, in view of such a situation, the present inventors have created the embodiments of the present disclosure configured to prevent falsification of the number of steps. In the embodiments of the present disclosure described below, the reliability of the number of steps measured that is measured using various sensing data about a user, such as behavior recognition, is calculated, and whether to adopt the number of steps upon calculating the insurance premium is determined depending on the reliability. Furthermore, in the present embodiment, in order to prevent falsification, it is preferable to perform personal authentication.

The embodiments of the present disclosure described below is applied to information processing for acquiring the number of steps for health promotion insurance. Note that the present embodiment is not limited to application to the information processing for acquiring the number of steps for calculation of the insurance premium of the health amplification insurance or for the discount (repayment). For example, the present embodiment may be applied to, for example, a service of giving discount points (incentive) that can be used for shopping or the like instead of cash to the user depending on the number of steps, or a service of giving health advice to the user depending on the number of steps.

Note that, in the present description, “the number of steps” includes not only the number of steps in user's walking but also the number of steps in user's running. In other words, “the number of steps” in the present description can be said to be the number of times of user's stepping during the body movement of him-/her-self.

### 2. EMBODIMENTS

### <2.1 Overview of Information Processing System 10 According to Embodiment of Present Disclosure>

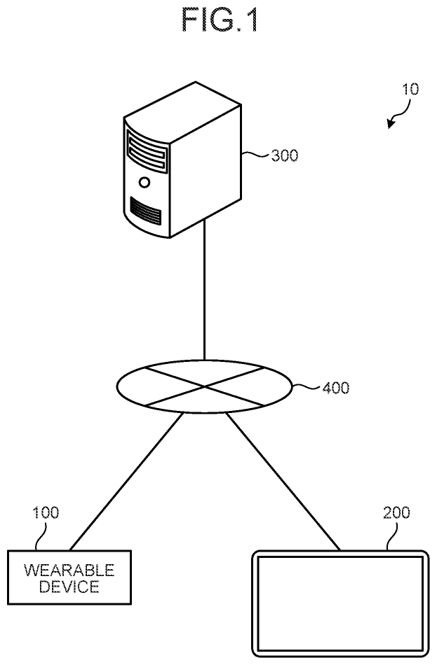

First, an outline of an information processing system 10 according to an embodiment of the present disclosure will be described with reference to FIG. 1. FIG. 1 is an illustrative diagram illustrating an exemplary configuration of the information processing system 10 according to the present embodiment.

As illustrated in FIG. 1, the information processing system 10 according to the present embodiment includes a wearable device 100, a mobile device 200, and a server 300, which are communicably connected to each other via a network 400. Specifically, the wearable device 100, the mobile device 200, and the server 300 are configured to be connected to the network 400 via a base station or the like (e.g., a mobile phone base station, a wireless local area network (LAN) access point, etc.) which is not illustrated. Note that, as a communication method used for the network 400, any method can be applied regardless of wired or wireless (e.g., WiFi (registered trademark), Bluetooth (registered trademark), etc.), but it is desirable to use a communication method capable of maintaining a stable operation. Hereinafter, an outline of each device included in the information processing system 10 according to the present embodiment will be sequentially described.

### (Wearable Device 100)

The wearable device 100 can be a device that is configured to be worn on a part of the user's body (earlobe, neck, arm, wrist, ankle, etc.) or an implant device (implant terminal) inserted into the user's body. More specifically, the wearable device 100 can be wearable devices of various types, such as a head mounted display (HMD) type, spectacle type, ear device type, anklet type, bracelet (wristband) type, collar type, eyewear type, pad type, batch type, and clothing type. Furthermore, the wearable device 100 includes, for example, a plurality of sensors including a sensor detecting a pulse wave signal from the user's pulse. Note that, in the following description, the wearable device 100 is assumed to be, for example, a bracelet (wristband) wearable device. Furthermore, details of the wearable device 100 will be described later.

### (Mobile Device 200)

The mobile device 200 is an information processing terminal carried by the user. Specifically, the mobile device 200 is configured to receive information input from the user and sensing data from the wearable device 100, process the received information and the like, and output the processed information and the like to the server 300 which is described later. For example, the mobile device 200 can be a device such as a tablet personal computer (PC), smartphone, mobile phone, laptop PC, notebook PC, or HMD. Furthermore, the mobile device 200 includes a display unit (not illustrated) that performs display for the user, an input unit (not illustrated) that receives an input operation from the user, a speaker (not illustrated) that outputs voice to the user, a microphone (not illustrated) that acquires surrounding voice, and the like. Note that, in the following description, the mobile device 200 is assumed to be, for example, a smartphone. Furthermore, details of the mobile device 200 will be described later.

Note that, in the present embodiment, the mobile device 200 may be provided with various sensors included in the wearable device 100 described above, or the sensors may be provided separately from the wearable device 100 and the mobile device 200.

### (Server 300)

The server 300 includes, for example, a computer and the like. For example, the server 300 processes sensing data or information acquired by the wearable device 100 or the mobile device 200, and outputs information obtained by the processing to another device (e.g., mobile device 200). Specifically, for example, the server 300 is configured to process data about the number of steps obtained by processing the sensing data from the wearable device 100 by the mobile device 200 to calculate the insurance premium (e.g., discount amount etc.) (incentive). Furthermore, the server 300 is configured to output the calculated insurance premium to the mobile device 200. Note that details of the server 300 will be described later.

Note that, in FIG. 1, the information processing system 10 according to the present embodiment is illustrated as including one wearable device 100 and one mobile device 200, but the present embodiment is not limited thereto. For example, the information processing system 10 according to the present embodiment may include a plurality of wearable devices 100 and mobile devices 200. Furthermore, the information processing system 10 according to the present embodiment may include, for example, another communication device, such as a relay device, used upon transmission of information from the wearable device 100 or the mobile device 200 to the server 300.

Furthermore, the information processing system 10 according to the present embodiment may not include the wearable device 100. In such a configuration, for example, the mobile device 200 may function as the wearable device 100, and sensing data acquired by the mobile device 200 or information obtained by processing the sensing data may be output to the server 300.

### <2.2 Detailed Configuration of Wearable Device 100>

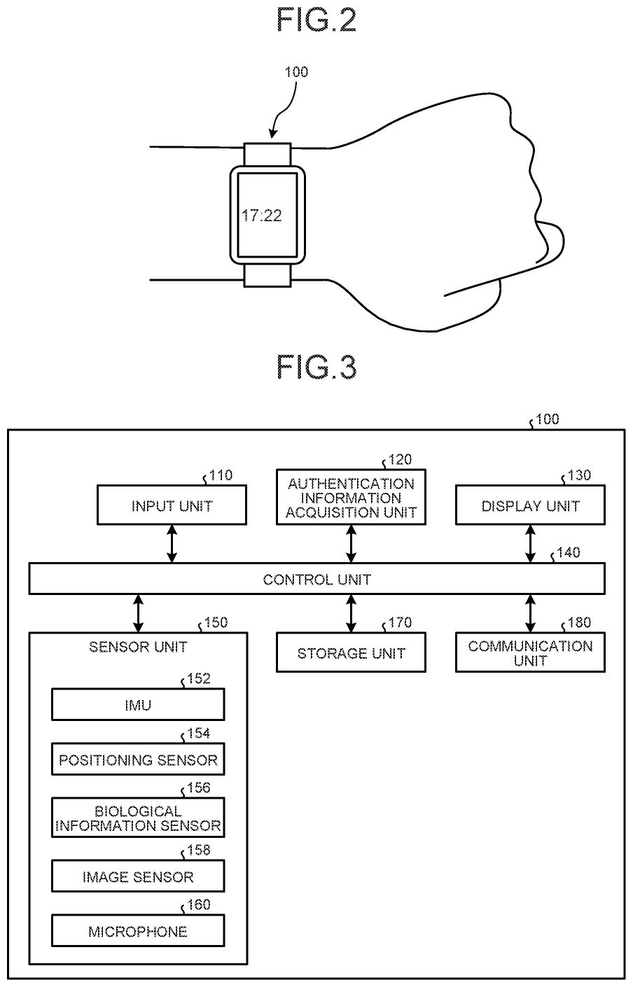

Next, a detailed configuration of the wearable device 100 according to an embodiment of the present disclosure will be described with reference to FIGS. 2 and 3. FIG. 2 is an illustrative diagram illustrating an exemplary appearance of the wearable device 100 according to the present embodiment, and FIG. 3 is a block diagram illustrating an exemplary configuration of the wearable device 100 according to the present embodiment.

As described above, as the wearable device 100, the wearable devices of various types, such as a bracelet type and an HMD type can be adopted. FIG. 2 illustrates the exemplary appearance of the wearable device 100 according to the present embodiment. As illustrated in FIG. 2, the wearable device 100 is a bracelet type wearable device worn on a wrist of the user.

Specifically, as illustrated in FIG. 2, the wearable device 100 includes a strap-shaped band portion. For example, the band portion is worn so as to be wound around the wrist of the user, and therefore, the band portion is formed of a material such as soft silicone gel so as to have a ring shape in conformity with the shape of the wrist. Furthermore, a control unit (not illustrated) is a portion in which the sensors and the like described above are provided, and is provided inside the band portion so as to make contact with an arm of the user when the wearable device 100 is worn on the arm of the user.

Furthermore, as illustrated in FIG. 3, the wearable device 100 mainly includes an input unit 110, an authentication information acquisition unit 120, a display unit 130, a control unit 140, a sensor unit 150, a storage unit 170, and a communication unit 180. Details of functional units of the wearable device 100 will be sequentially described below.

### (Input Unit 110)

The input unit 110 receives inputs of data and a command from the user to the wearable device 100. More specifically, the input unit 110 is implemented by a touch screen, a button, a microphone, or the like. Furthermore, in the present embodiment, the input unit 110 may be, for example, a gaze sensor that detects the line of sight of the user and receives a command associated with a display the user is gazing. The gaze sensor can be implemented by, for example, an imaging device including a lens, an imaging element, and the like. Furthermore, the input unit 110 may be an input unit that receives an input by detecting a gesture of a hand or arm on which the wearable device 100 is worn, by using an inertial measurement unit (IMU) 152 included in the sensor unit 150 which is described later.

### (Authentication Information Acquisition Unit 120)

The authentication information acquisition unit 120 is configured to acquire a fingerprint pattern image, an iris pattern image, vein pattern image, or face image of the user, a voiceprint based on the user's voice, or the like, in order to perform personal authentication for the user, and the acquired information is transmitted to the mobile device 200 which is described later. Furthermore, in the present embodiment, the authentication information acquisition unit 120 may receive a password, a trajectory shape, and the like input from the user for the personal authentication for the user.

In the present embodiment, for example, in a case where the personal authentication is performed on the basis of fingerprint information of the user, the authentication information acquisition unit 120 can be a capacitive fingerprint sensor that acquires a fingerprint pattern by sensing the capacitance at each point on a sensing surface generated when a fingertip of the user is placed on the sensing surface. The capacitive fingerprint sensor is configured to detect a fingerprint pattern by applying a small current to microelectrodes arranged in a matrix on the sensing surface to detect a potential difference appearing in capacitance generated between the microelectrode and the fingertip.

Furthermore, in the present embodiment, the authentication information acquisition unit 120 may be, for example, a pressure fingerprint sensor that acquires the fingerprint pattern by sensing pressure at each point on the sensing surface generated when the fingertip is placed on the sensing surface. In the pressure fingerprint sensor, for example, semiconductor micro-sensors whose resistance values change depending on pressure are arranged in matrix on the sensing surface.

Furthermore, in the present embodiment, the authentication information acquisition unit 120 may be, for example, a thermal fingerprint sensor that acquires the fingerprint pattern by sensing a temperature difference generated when the fingertip is placed on the sensing surface. In the thermal fingerprint sensor, for example, temperature micro-sensors whose resistance values change depending on temperature are arranged in matrix on the sensing surface.

Furthermore, in the present embodiment, the authentication information acquisition unit 120 may be, for example, an optical fingerprint sensor that acquires a captured image of a fingerprint pattern by detecting reflected light generated when the fingertip is placed on the sensing surface. The optical fingerprint sensor includes, for example, a micro lens array (MLA) which is an example of a lens array, and a photoelectric conversion element. In other words, the optical fingerprint sensor can be said to be one type of imaging device.

Furthermore, in the present embodiment, the authentication information acquisition unit 120 may be, for example, an ultrasonic fingerprint sensor that acquires a fingerprint pattern by emitting an ultrasonic wave and detecting ultrasonic waves reflected on uneven skin surface of the fingertip.

### (Display Unit 130)

The display unit 130 is a device for presenting information to the user, and, for example, outputs various information to the user by using images. More specifically, the display unit 130 is implemented by a display or the like. Note that some of the functions of the display unit 130 may be provided by the mobile device 200. Furthermore, in the present embodiment, a functional block that presents information to the user is not limited to the display unit 130, and the wearable device 100 may have a functional block such as a speaker, earphone, light emitting element (e.g., light emitting diode (LED)), or vibration module.

### (Control Unit 140)

The control unit 140 is provided in the wearable device 100 so as to control each functional unit of the wearable device 100 and acquire sensing data from the sensor unit 150 described above. The control unit 140 is implemented by hardware such as a central processing unit (CPU), a read only memory (ROM), and a random access memory (RAM). Note that some of the functions of the control unit 140 may be provided by the server 300 which is described later.

### (Sensor Unit 150)

The sensor unit 150 is provided in the wearable device 100 worn on the body of the user, includes various sensors that detect the conditions of the user or the conditions of the surrounding environment of the user, and transmits the sensing data acquired by these various sensors to the mobile device 200 which is described later. Specifically, the sensor unit 150 includes the inertial measurement unit (IMU) 152 that detects inertial data generated by the movement of the user, a positioning sensor 154 that measures a position of the user, and a biological information sensor 156 that detects the pulse or heart rate of the user. Furthermore, the sensor unit 150 can include an image sensor 158 that acquires an image (moving image) around the user, one or a plurality of microphones 160 that detects environmental sound around the user, and the like. Details of various sensors of the sensor unit 150 will be described below.

˜IMU 152˜

The IMU 152 is configured to acquire sensing data (inertial data) indicating a change in acceleration or angular velocity that occurs with the motion of the user. Specifically, the IMU 152 includes an acceleration sensor, a gyro sensor, a geomagnetic sensor, and the like (not illustrated).

### ˜Positioning Sensor 154˜

The positioning sensor 154 is a sensor that detects the position of the user wearing the wearable device 100, and can be specifically a global navigation satellite system (GNSS) receiver or the like. In this configuration, the positioning sensor 154 can generate sensing data indicating the latitude and longitude of the current location of the user on the basis of a signal (GNSS signal) from a GNSS satellite. Furthermore, in the present embodiment, it is possible to detect a relative positional relationship of the user from, for example, information about radio frequency identification (RFID), a Wi-Fi access point, a radio base station, and the like, and therefore, such a communication device can also be used as the positioning sensor 154.

Note that, in the present embodiment, a proof of location (PoL) technology may also be used to increase the reliability of positioning by the GNSS signal. For example, the PoL technology is a technology that performs short-range communication with a fixed access point in the vicinity of a position determined by the GNSS signal, simultaneously with positioning by the GNSS signal, confirming that the user is positioned at the position, increasing the reliability of positioning by the GNSS signal.

### ˜Biological Information Sensor 156˜

The biological information sensor 156 is a sensor that detects biological information of the user, and can be, for example, various sensors that are directly attached to a part of the user's body to measure the user's heart rate, pulse, blood pressure, brain wave, respiration, perspiration, myoelectric potential, skin temperature, electrical skin resistance, and the like.

For example, a heart rate sensor (example of pulse sensor) is a sensor that detects the heart rate that is a pulsation of the heart of the user. In addition, a pulse sensor (example of the pulse sensor) is a sensor that detects the pulse on a body surface or the like, the pulse being a pulsation of an artery caused by a change in pressure on an inner wall of the artery due to sending of blood to the whole body through the arteries by the pulsation (heart rate) of the heart. Furthermore, a blood flow sensor (including blood pressure sensor) is, for example, a sensor that emits infrared rays or the like to the body to obtain the absorptivity or reflectance of light or the change thereof and detects the blood flow rate, pulse, heart rate, and blood pressure. Furthermore, the heart rate sensor or the pulse sensor may be an imaging device that images the skin of the user. In this case, the pulse or heart rate of the user can be detected on the basis of a change in light reflectance on the skin obtained from the image of the skin of the user.

For example, a respiration sensor can be a respiratory flow sensor that detects a change in respiration. A brain wave sensor is a sensor that detects a brain wave by removing noise from a change in potential difference between a plurality of electrodes measured by attaching the electrodes to a scalp of the user to extract periodic waves. A skin temperature sensor is a sensor that detects the surface temperature of the user, and a skin conductivity sensor is a sensor that detects the electrical skin resistance of the user. A perspiration sensor is a sensor that is worn on the skin of the user to detect the change in voltage or resistance between two points on the skin, caused by perspiration. Furthermore, a myoelectric sensor is a sensor that quantitatively detects a muscle activity of a muscle by measuring a myoelectric potential. The myoelectric potential is measured based on an electric signal that is generated in a muscle fiber when a muscle of an arm or the like is contracted and that is propagated to a body surface, with a plurality of electrodes attached to the arm or the like of the user.

### ˜Image Sensor 158˜

The image sensor 158 is, for example, an image sensor for color imaging that has a Bayer array capable of detecting blue, green, and red light. Furthermore, this RGB sensor may include a pair of image sensors in order to recognize a depth (stereo system).

Furthermore, the image sensor 158 may be a time of flight (ToF) sensor that acquires depth information of a real space around the user. Specifically, the ToF sensor emits irradiation light such as infrared light around the user to detect reflected light reflected from the surface of an object around the user. Then, the ToF sensor calculates a phase difference between the irradiation light and the reflected light to acquire a distance (depth information) from the ToF sensor to a real object. Therefore, it is possible to obtain a distance image as three-dimensional shape data, from such depth information. Note that the method of obtaining distance information based on the phase difference as described above is referred to as an indirect ToF method. Furthermore, in the present embodiment, it is also possible to use a direct ToF method in which a round-trip time from emission of light to reception of the light reflected from the object is detected to acquire the distance (depth information) from the ToF sensor to the object. In detail, the ToF sensor is configured to acquire the distance (depth information) from the ToF sensor to the object, and therefore, the distance image including the distance information (depth information) indicating the distance to the object can be obtained as the three-dimensional shape data of the real space. Here, the distance image is, for example, image information generated by associating the distance information (depth information) acquired for each pixel of the ToF sensor with position information of the corresponding pixel.

˜Microphone 160˜

The microphone 160 is a sound sensor that detects a sound generated by speech voice or motion of the user or a sound generated around the user. Note that, in the present embodiment, the number of microphones 160 is not limited to one, and a plurality of the microphones 160 may be employed. Furthermore, in the present embodiment, the microphone 160 is provided in the wearable device 100, but the microphone 160 is not limited to this description, and one or more microphones 160 may be installed around the user.

Furthermore, the sensor unit 150 may include an ambient environment sensor that detects the conditions of the surrounding environment of the user, and specifically, may include various sensors that detect the temperature, humidity, brightness, and the like of the surrounding environment of the user. In the present embodiment, the sensing data from these sensors may be used to improve the accuracy in recognition of user's behaviors which are described later.

Furthermore, the sensor unit 150 may incorporate a clock mechanism (not illustrated) that provides the accurate time associate the acquired sensing data with the time at which the sensing data is acquired. Furthermore, as described above, the various sensors may not be provided in the sensor unit 150 of the wearable device 100. For example, the various sensors may be provided separately from the wearable device 100, or may be provided in another device or the like used by the user.

Furthermore, the sensor unit 150 may include a sensor for detecting a mounted state of the sensor unit 150. For example, the sensor unit 150 may include a pressure sensor or the like that detects appropriate mounting of the sensor unit 150 on a part of the body of the user (e.g., mounting thereof in close contact with the part of the body).

### (Storage Unit 170)

The storage unit 170 is provided in the wearable device 100 to store programs, information, and the like for the control unit 140 to execute various processing, and information obtained by the processing. Note that the storage unit 170 is implemented by a nonvolatile memory or the like such as a flash memory.

### (Communication Unit 180)

The communication unit 180 is provided in the wearable device 100 so as to transmit and receive information to and from an external device, such as the mobile device 200 or the server 300. In other words, it can be said that the communication unit 180 is a communication interface having a function of transmitting and receiving data. Note that the communication unit 180 is implemented by communication devices such as a communication antenna, a transmission/reception circuit, and a port. Furthermore, in the present embodiment, the communication unit 180 may be caused to function as a radio wave sensor that detects a radio field intensity or radio arrival direction.

Note that, in the present embodiment, the configuration of the wearable device 100 is not limited to that illustrated in FIG. 3, and may further include, for example, a functional block not illustrated or the like.

### <2.3 Detailed Configuration of Mobile Device 200>

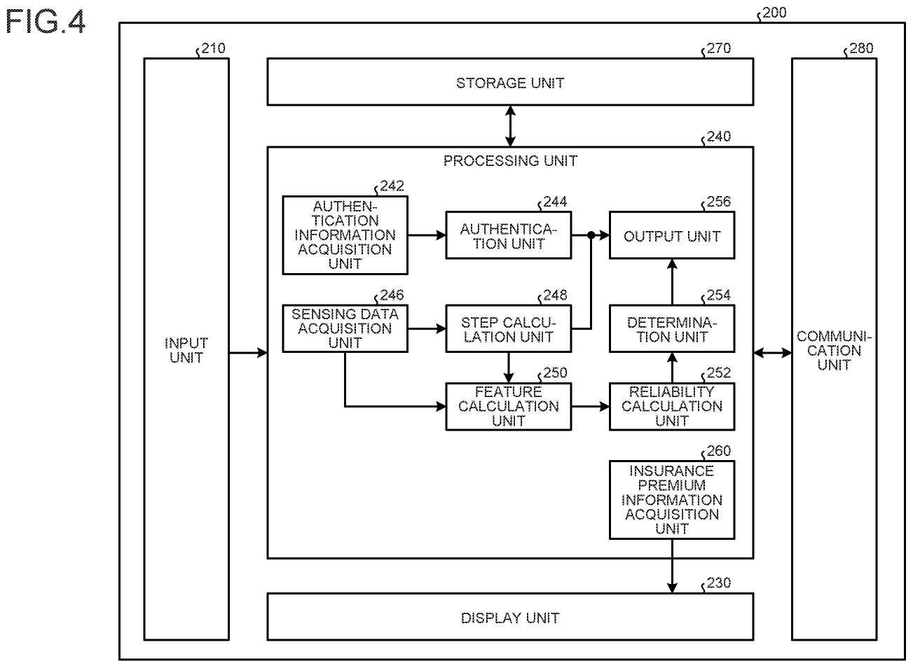

Next, a detailed configuration of the mobile device 200 according to the present embodiment will be described with reference to FIG. 4. FIG. 4 is a block diagram illustrating an exemplary configuration of the mobile device 200 according to the present embodiment. As described above, the mobile device 200 is a device such as a tablet, smartphone, mobile phone, laptop PC, notebook PC, or HMD. Specifically, as illustrated in FIG. 4, the mobile device 200 mainly includes an input unit 210, a display unit 230, a processing unit 240, a storage unit 270, and a communication unit 280. Details of functional units of the mobile device 200 will be sequentially described below.

### (Input Unit 210)

The input unit 210 receives inputs of data and a command from the user to the mobile device 200. More specifically, the input unit 210 is implemented by a touch screen, a button, a microphone, and the like.

### (Display Unit 230)

The display unit 230 is a device for presenting information to the user, and, for example, is configured to output various information to the user by using images based on information acquired from the server 300. More specifically, the display unit 130 is implemented by a display or the like. Furthermore, in the present embodiment, a functional block that presents information to the user is not limited to the display unit 230, and the mobile device 200 may have a functional block such as a speaker, earphone, light emitting element, or vibration module.

### (Processing Unit 240)

The processing unit 240 is configured to process the sensing data from the sensor unit 150 of the wearable device 100. The processing unit 240 is implemented by hardware such as CPU, ROM, and RAM. As illustrated in FIG. 4, the processing unit 240 includes an authentication information acquisition unit 242, an authentication unit 244, a sensing data acquisition unit 246, a step calculation unit (calculation unit) 248, a feature calculation unit 250, a reliability calculation unit 252, a determination unit 254, an output unit 256, and an insurance premium information acquisition unit (presentation unit) 260. Details of functional blocks of the processing unit 240 will be sequentially described below.

### ˜Authentication Information Acquisition Unit 242˜

The authentication information acquisition unit 242 is configured to acquire, from the authentication information acquisition unit 120 of the wearable device 100, the fingerprint pattern image, iris pattern image, vein pattern image, or face image of the user, the voiceprint based on the user's voice, or the like, in order to perform personal authentication for the user. Furthermore, the authentication information acquisition unit 242 is configured to output the acquired information to the authentication unit 244 which is described later. In the present embodiment, for example, in a case where the personal authentication is performed on the basis of the fingerprint information of the user, the authentication information acquisition unit 242 may acquire the fingerprint pattern of the user from the authentication information acquisition unit 120 of the wearable device 100 to perform predetermined processing for emphasizing the fingerprint pattern, removing noise, and the like. More specifically, the authentication information acquisition unit 242 is configured to use various filters for smoothing and removing noise, such as, a moving average filter, difference filter, median filter, and Gaussian filter. Furthermore, the authentication information acquisition unit 242 may perform processing by using, for example, various algorithms for binarization and thinning.

### ˜Authentication Unit 244˜

The authentication unit 244 is configured to collate, in advance, the fingerprint information (fingerprint pattern), iris information, face image, password, trajectory, and the like of the user acquired from the authentication information acquisition unit 242 described above, with personal authentication information associated with a personal identification (ID) in a personal information database (DB) stored in the storage unit 270 which is described later, for personal authentication.

In the present embodiment, for example, when personal authentication is performed based on the fingerprint information of the user, the authentication unit 244 calculates features of the fingerprint pattern. Here, the features of the fingerprint pattern refer to a distribution of the feature points on the fingerprint pattern, that is, the number of feature points or a distribution density (distribution information) of the feature points. Furthermore, the feature points refer to attribute information such as the shapes, directions, and positions (relative coordinates) of a center point of the fingerprint pattern, and a branch point, intersection point, and end point of a ridge (referred to as minutiae) of a pattern of the fingerprint pattern. Furthermore, the feature points may be attribute information such as the shapes, directions, widths, interval, and distribution density of the ridges.

Then, for example, the authentication unit 244 is also configured to collate feature points extracted from part of the fingerprint pattern output from the authentication information acquisition unit 242 with feature points of the fingerprint pattern recorded in the storage unit 130 or the like in advance, for authentication of the user (minutiae method). Furthermore, for example, the authentication unit 244 is configured to collate the fingerprint pattern output from the authentication information acquisition unit 242 described above with a fingerprint template of the fingerprint pattern stored in the storage unit 270 or the like in advance, for authentication of the user (pattern matching method). Furthermore, for example, the authentication unit 244 is also configured to perform spectral analysis of a pattern for each sliced fingerprint pattern obtained by slicing the fingerprint pattern into strips, and perform collation by using a result of spectral analysis of the fingerprint pattern stored in advance in the storage unit 270 or the like, for performing authentication.

In the present embodiment, when the personal authentication of the user is successfully performed by the authentication unit 244, it is possible to start acquisition of the sensing data, process the sensing data, and transmit data obtained by processing the sensing data to an external device (e.g., the server 300).

### ˜Sensing Data Acquisition Unit 246˜

The sensing data acquisition unit 246 is configured to acquire a plurality of pieces of sensing data from the wearable device 100 and output the sensing data to the step calculation unit 248 and the feature calculation unit 250 which are described later.

### ˜Step Calculation Unit 248˜

The step calculation unit 248 is configured to calculate (count) the number of steps of the user on the basis of a change in the inertial data (acceleration data, angular velocity data, etc.) from the sensing data acquisition unit 246 described above. Note that the step calculation unit 248 may calculate the number of steps of the user, referring to a model obtained in advance by machine learning. Furthermore, the step calculation unit 248 is configured to output data about the number of steps calculated, to the output unit 256 which is described later, and the like. Note that in the present embodiment, the step calculation unit 248 may calculate a movement distance of the user.

### ˜Feature Calculation Unit 250˜

The feature calculation unit 250 is configured to calculate the features from the inertial data, position data, biological data, and environment data included in the plurality of pieces of sensing data from the sensing data acquisition unit 246 described above (details of the inertial data, position data, biological data, and environment data will be described later). Furthermore, the feature calculation unit 250 is configured to output the calculated features to the reliability calculation unit 252 which is described later. For example, the feature calculation unit 250 is configured to calculate the features by performing statistical processing (average, variance, normalization, etc.) on one or more of the plurality of pieces of sensing data. Alternatively, the feature calculation unit 250 may calculate the features from the sensing data, referring to a model obtained in advance by machine learning.

Furthermore, for example, the feature calculation unit 250 is also configured to obtain a walking distance of the user (second distance data) by multiplying the data about the number of walking steps of the user obtained from the inertial data, and data about a stride of the user input from the user. Furthermore, the feature calculation unit 250 may calculate a distance (first distance data) through which the user has moved by walking on the basis of the sensing data from the positioning sensor 154 and calculate, as the features, a difference between a walking distance based on the inertial data and a walking distance based on the sensing data from the positioning sensor 154.

Furthermore, the feature calculation unit 250 is configured to recognize the user's behaviors (walking, running, driving, etc.) as the features, on the basis of at least one of the plurality of pieces of sensing data from the sensing data acquisition unit 246 described above. For example, in a case where the same type of sensor (e.g., IMU 152) is mounted on both of the wearable device 100 and the mobile device 200, the feature calculation unit 250 is configured to compare the same type of sensing data (inertial data) from different devices to recognize the user's behaviors. Note that details of calculation of the features in the present embodiment will be described later.

### ˜Reliability Calculation Unit 252˜

The reliability calculation unit 252 is configured to calculate the reliability on the basis of the features obtained from the position data, biological data, environment data, and behavior recognition data about the user obtained by the feature calculation unit 250 described above. Furthermore, the reliability calculation unit 252 is configured to output the calculated reliability to the determination unit 254 which is described later. Specifically, the reliability calculation unit 252 is configured to calculate the reliability by weighting each of the features with a predetermined coefficient given to each feature. Furthermore, in the present embodiment, the reliability calculation unit 252 may dynamically change the predetermined coefficient according to the position, behavior recognition data, a change in position, and the like about the user. Note that details of the calculation of the reliability in the present embodiment will be described later.

### ˜Determination Unit 254˜

The determination unit 254 is configured to determine whether to accept the data about the number of steps calculated by the step calculation unit 248 (specifically, whether the number of steps is true or false) on the basis of the reliability calculated by the reliability calculation unit 252 described above. Specifically, the determination unit 254 compares the reliability with a predetermined threshold. For example, when the reliability is equal to or above the predetermined threshold, it is determined to receive the data about the number of steps calculated, and the result of determination is output to the output unit 256 which is described later. Furthermore, in the present embodiment, the determination unit 254 may dynamically change the predetermined threshold on the basis of information from the server 300.

### ˜Output Unit 256˜

The output unit 256 outputs the data about the number of steps calculated by the step calculation unit 248 to the server 300, on the basis of the determination by the determination unit 254 described above. Note that the output unit 256 may output the data about the number of steps to the display unit 230 or the storage unit 270.

### ˜Insurance Premium Information Acquisition Unit 260˜

In the server 300, the insurance premium information acquisition unit 260 is configured to acquire information about the insurance premium or discount amount (incentive) of the insurance premium that is calculated on the basis of the data about the number of steps, from the server 300 and output the information to the display unit 230.

### (Storage Unit 270)

The storage unit 270 is provided in the mobile device 200 to store programs, information, and the like for the above-described processing unit 240 to perform various processing, and information obtained by the processing. Note that the storage unit 270 is implemented by a nonvolatile memory or the like such as a flash memory.

### (Communication Unit 280)

The communication unit 280 is provided in the mobile device 200 so as to transmit and receive information to and from an external device such as the wearable device 100 or the server 300. In other words, it can be said that the communication unit 280 is a communication interface having a function of transmitting and receiving data. Note that the communication unit 280 is implemented by communication devices such as a communication antenna, a transmission/reception circuit, and a port. Furthermore, in the present embodiment, the communication unit 280 may be caused to function as a radio wave sensor that detects a distance to the wearable device 100 or that detects a radio field intensity or radio arrival direction.

Note that, in the present embodiment, the configuration of the mobile device 200 is not limited to that illustrated in FIG. 4, and may further include, for example, an unillustrated functional block or the like such as the sensor unit 150 of the wearable device 100.

### <2.4 Detailed Configuration of Server 300>

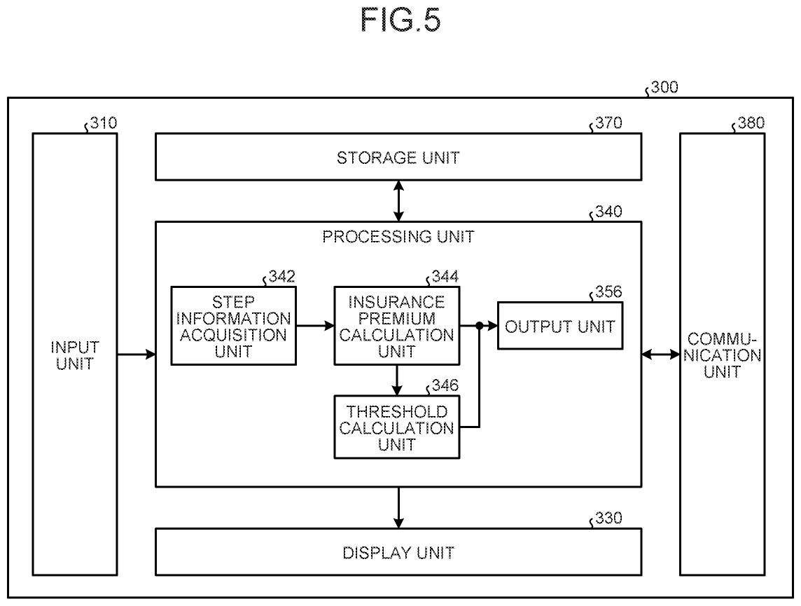

Next, a detailed configuration of the server 300 according to the present embodiment will be described with reference to FIG. 5. FIG. 5 is a block diagram illustrating an exemplary configuration of the server 300 according to the present embodiment. As described above, the server 300 includes, for example, the computer or the like. Specifically, as illustrated in FIG. 5, the server 300 mainly includes an input unit 310, a display unit 330, a processing unit 340, a storage unit 370, and a communication unit 380. Details of functional units of the server 300 will be sequentially described below.

### (Input Unit 310)

The input unit 310 receives inputs of data and a command from the user to the server 300. More specifically, the input unit 310 is implemented by a touch screen, a keyboard, and the like.

### (Display Unit 330)

The display unit 330 includes, for example, a display, a video output terminal, and the like and outputs various information to the user by using images or the like.

### (Processing Unit 340)

The processing unit 340 is provided in the server 300 to control each block of the server 300. Specifically, the processing unit 340 controls various processing such as calculation of the insurance premium performed in the server 300. The processing unit 340 is implemented by hardware such as CPU, ROM, and RAM. Note that the processing unit 340 may perform some of the functions of the processing unit 240 of the mobile device 200. Specifically, as illustrated in FIG. 5, the processing unit 340 includes a step information acquisition unit 342, an insurance premium calculation unit 344, a threshold calculation unit 346, and an output unit 356. Details of functional blocks of the processing unit 340 will be sequentially described below.

### ˜Step Information Acquisition Unit 342˜

The step information acquisition unit 342 is configured to acquire the data about the number of steps from the mobile device 200 and output the data to the insurance premium calculation unit 344 and the storage unit 370 which are described later.

### ˜Insurance Premium Calculation Unit 344˜

The insurance premium calculation unit 344 is configured to calculate the insurance premium of the user on the basis of the data about the number of steps from the step information acquisition unit 342 described above and output the calculated insurance premium to the output unit 356 which is described later. Specifically, the insurance premium calculation unit 344 is configured to calculate the insurance premium of the user, on the basis of the number of steps of the user and the attribute information (gender, age, places where the user has lived, medical history, occupation, desired compensation, etc.) of the user, referring to an insurance premium table stored in the storage unit 370 which is described later. At this time, the insurance premium calculation unit 344 may also calculate and output a difference (discount amount) between the current insurance premium of the user and a newly calculated insurance premium.

### ˜Threshold Calculation Unit 346˜

The threshold calculation unit 346 is configured to determine the predetermined threshold used by the determination unit 254 of the mobile device 200 described above, referring to a past history (the number of steps or the like) of the user (a plurality of users) and the results of the guarantee and management of the insurance company and output the predetermined threshold to the mobile device 200 via the output unit 356 which is described later. Specifically, for example, the threshold calculation unit 346 adjusts the threshold so that the insurance company can achieve a benefit even when the insurance premium is reduced for each user according to the result of the number of steps of each user.

### ˜Output Unit 356˜

The output unit 356 is configured to output the insurance premium and threshold calculated by the insurance premium calculation unit 344 and the threshold calculation unit 346 described above, to the mobile device 200.

### (Storage Unit 370)

The storage unit 370 is provided in the server 300 to store programs and the like for the processing unit 340 described above to perform various processing, and information obtained by the processing. More specifically, the storage unit 370 is implemented by a magnetic recording medium such as a hard disk (HD).

### (Communication Unit 380)

The communication unit 380 is provided in the server 300 so as to transmit and receive information to and from an external device such as the mobile device 200. Note that the communication unit 380 is implemented by, for example, communication devices such as a communication antenna, a transmission/reception circuit, and a port.

Note that, in the present embodiment, the configuration of the server 300 is not limited to that illustrated in FIG. 5, and for example, may further include, for example, an unillustrated functional block or the like that plays a part in the functions of the mobile device 200 described above.

### <2.5 Information Processing Method>

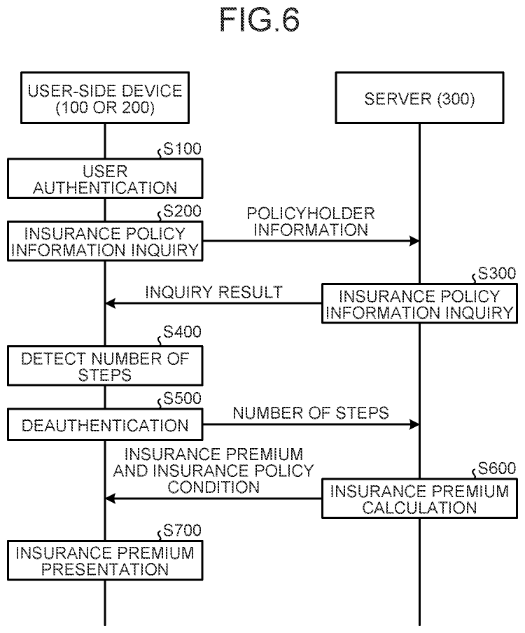

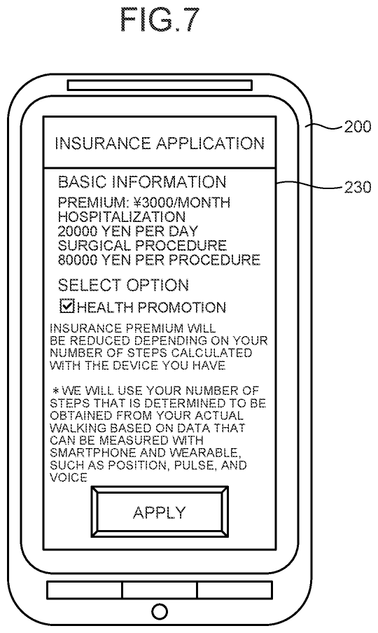

Next, an information processing method according to an embodiment of the present disclosure will be described with reference to FIGS. 6 to 15. FIG. 6 is a sequence diagram illustrating an exemplary information processing method according to the present embodiment, and FIGS. 7 to 15 are each an illustrative diagram illustrating an exemplary display screen according to the present embodiment.

Specifically, as illustrated in FIG. 6, the information processing method according to the present embodiment can mainly include a plurality of steps from Step S100 to Step S700. Details of these steps according to the present embodiment will be sequentially described below.

First, the wearable device 100 or the mobile device 200, which are user-side devices, performs personal authentication of the user (Step S100). For example, in Step S100, the mobile device 200 performs unlocking using the fingerprint pattern of the fingertip of the user.

Furthermore, in the present embodiment, the authentication information such as the fingerprint pattern used in the unlocking described above and information about the user's insurance policy (insured person identification information, insurance content information, insurance premium, etc.) are stored in the server 300 in association with each other in advance, in the first use. For example, as illustrated in FIG. 7, the mobile device 200 presents an image (a homepage image of the insurance company or the like) for application for insurance, to the user after the first unlocking. Then, when there is an operation showing user's intention to apply for the insurance, the application information is transmitted to the server 300 together with the authentication information such as the fingerprint pattern, whereby enabling the association as described above. At this time, the user preferably installs an insurance application on the wearable device 100 or the mobile device 200. Furthermore, the image preferably specifies that the insurance premium is re-calculated by using only the number of walking steps determined to be valid to calculate the insurance premium. In addition, from the viewpoint of protection of the privacy of the user, it is preferable to clearly show, on the screen, that the sensing data obtained from various sensors is used.

Next, the wearable device 100 or the mobile device 200, which is the user-side device, transmits authentication information of the user or identification information (policyholder information) associated with the user to the server 300, and inquires for the policyholder information (Step S200).

Then, the server 300 confirms whether the policyholder information or the like transmitted from the wearable device 100 or the mobile device 200 matches policyholder information or the like stored in advance, and transmits a confirmation result as an inquiry result to the wearable device 100 or the mobile device 200 (Step S300).

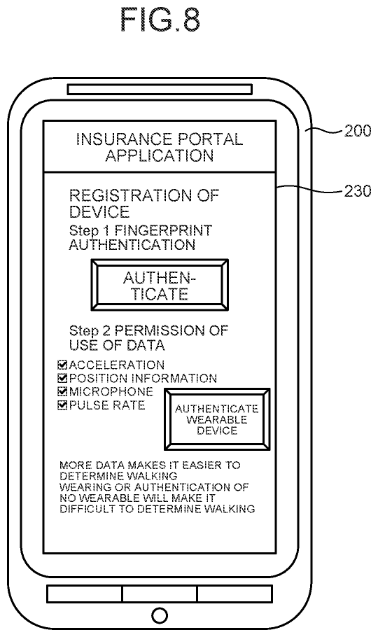

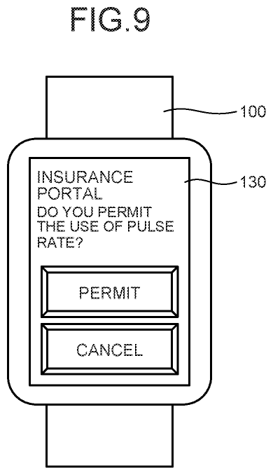

When the confirmation result transmitted from the server 300 indicates that the information matches each other, the wearable device 100 or the mobile device 200 starts to detect the number of steps (Step S400). On the other hand, when the confirmation result transmitted from the server 300 indicates that the information does not match each other, the wearable device 100 or the mobile device 200 finishes the processing. At this time, for example, as illustrated in FIGS. 8 and 9, the wearable device 100 or the mobile device 200 presents an image for requiring a user's approval for use of the sensing data, in order to start acquisition of the sensing data by various sensors mounted on the wearable device 100. Note that, when both of the wearable device 100 and the mobile device 200 are used, the wearable device 100 and the mobile device 200 are communicably connected through short-range communication or the like, and, as illustrated in FIG. 9, processing of requiring the approval for the use of the sensing data may be performed on the wearable device 100.

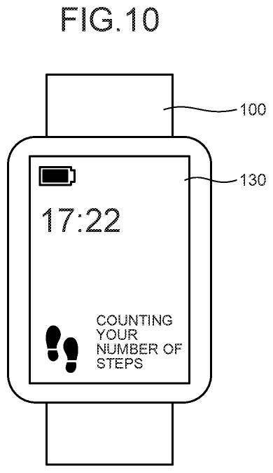

Furthermore, in the present embodiment, during the detection of the number of steps, for example, the wearable device 100 or the like may notify the user that the number of steps is being detected, as illustrated in FIG. 10.

Then, for example, when the wearable device 100 is dismounted or the short-range communication between the wearable device 100 and the mobile device 200 is interrupted, the wearable device 100 or the mobile device 200 deauthenticates the user (Step S500). Furthermore, upon deauthentication, the wearable device 100 or the mobile device 200 transmits data, such as the number of steps and the reliability, having been calculated to the server 300. Note that, in the present embodiment, the transmission is not limited to the transmission of data, such as the number of steps and the reliability, having been calculated, to the server 300 at the deauthentication timing, and may be, but not particularly limited to, transmission at the end of a day or every predetermined time period.

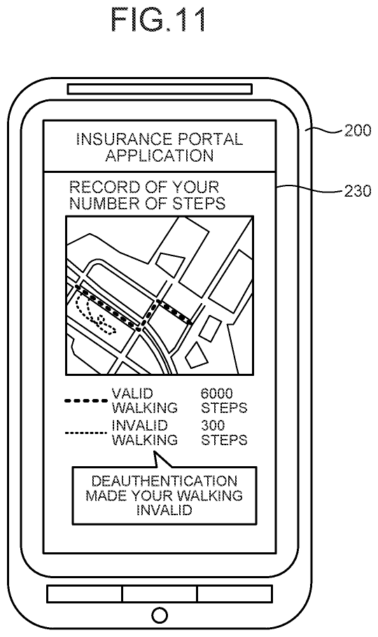

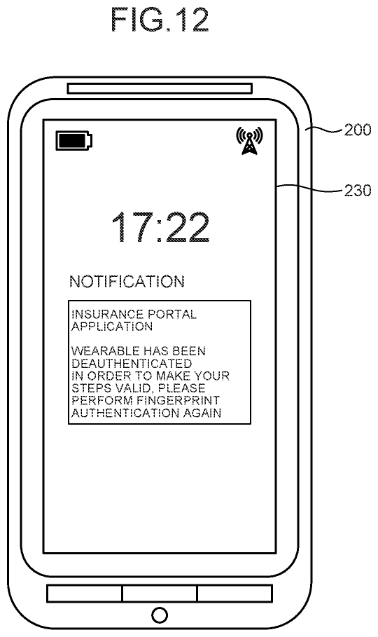

At this time, as illustrated in FIG. 11, for example, the mobile device 200 may present information about the number of steps valid and the number of steps invalid for calculating the insurance premium, together with a walking trajectory of the user shown on a map. In this manner, presenting the number of steps invalid, the reason for the invalidity, and the like to the user makes it possible for the user to confirm the number of steps reflected in the insurance premium later, improving the user's sense of satisfaction with the calculation of the insurance premium. In addition, upon deauthentication, a notification of the deauthentication and a notification of requiring re-authentication may be presented to the user, as illustrated in FIG. 12 Note that details of the number of steps, calculation of the reliability, and the like in the present embodiment will be described later.

Next, the server 300 calculates the insurance premium on the basis of data such as the number of steps transmitted from the wearable device 100 or mobile device 200 (Step S600). Then, the server 300 redefines and updates an insurance policy condition and the like of the user on the basis of the calculated insurance premium, and transmits information, such as the insurance premium (discount amount of the insurance premium) and the insurance policy condition, to the wearable device 100 or mobile device 200.

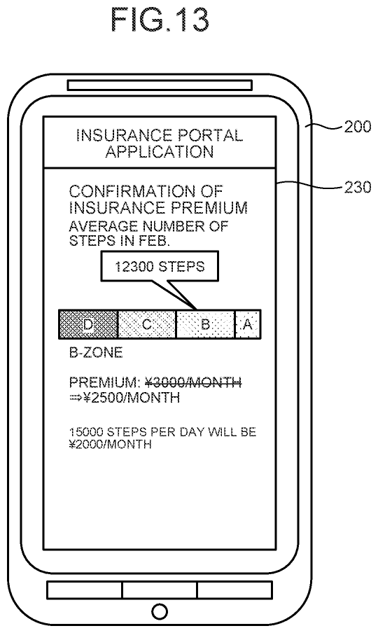

Then, the wearable device 100 or the mobile device 200 presents information, such as the insurance premium and the insurance policy condition, transmitted from the server 300 to the user (Step S700). For example, as illustrated in FIG. 13, the mobile device 200 may present a difference (discount amount) between the updated insurance premium and the insurance premium before the update together with the updated insurance premium.

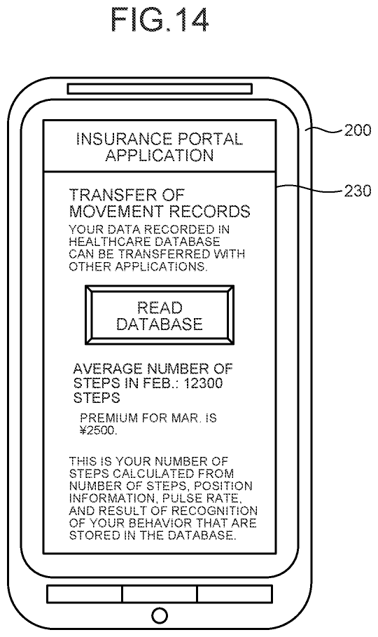

Furthermore, in the present embodiment, the wearable device 100 or the mobile device 200 may analyze past sensing data (e.g., sensing data acquired by the wearable device 100 before purchasing the insurance policy) that has already been stored to calculate the number of steps to be reflected in the insurance premium. Furthermore, in the present embodiment, the wearable device 100 or the mobile device 200 may have a standalone configuration to simulate or determine the insurance premium by using past data. At this time, for example, as illustrated in FIG. 14, the mobile device 200 may present a button for instruction to read the past data or a screen for display of the calculated insurance premium, to the user.

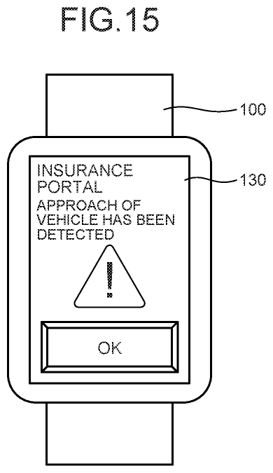

Furthermore, in the present embodiment, for example, when an environmental sound includes a sound (e.g., car horn or the like) with which danger to the user may be inferred where environmental sound around the user is acquired in order to acquire the features, the wearable device 100 may alert the user by giving a screen display, vibration, or the like to the user, as illustrated in FIG. 15. Furthermore, in the present embodiment, when falling of the user is inferred from the inertial data of the mobile device, or when taking away of the user by a suspicious person is inferred from voice data (e.g., the wording “Help” extracted from the voice data), the mobile device 200 may give automatic notification to a family member of the user, automatic request for rescue to an ambulance or police, or the like. The addition of such a function has an effect of prompting the user to approve constant acquisition of the sensing data using the various sensors.

### <2.6 Calculation of Number of Steps>

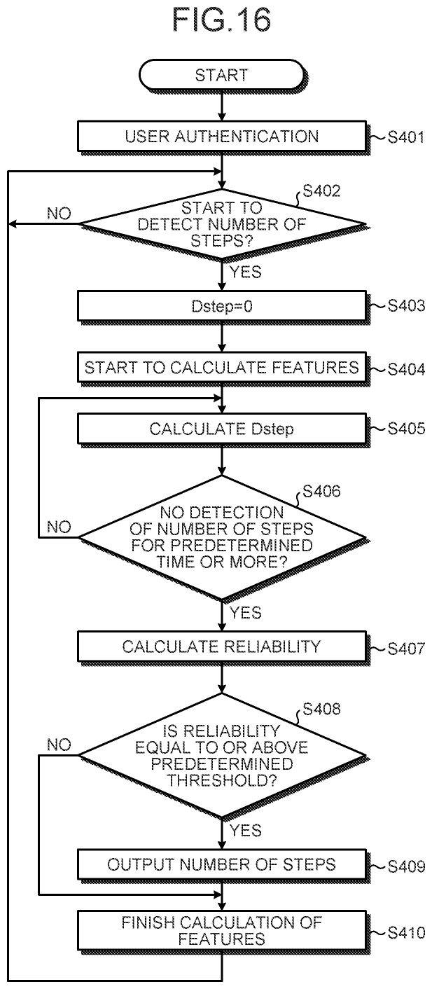

Next, details of calculation of the number of steps, illustrated in Step S400 of FIG. 6, will be described with reference to FIG. 16. FIG. 16 is a flowchart illustrating an exemplary information processing method according to the present embodiment. Specifically, as illustrated in FIG. 16, Step S400 of FIG. 6 may mainly include a plurality of substeps from Substep S401 to Substep S410. Details of these substeps according to the present embodiment will be sequentially described below.

First, as described in Step S100 of FIG. 6, personal authentication of the user is performed in the wearable device 100 or the mobile device 200 which is the user-side device (Substep S401).

The wearable device 100 or the mobile device 200 determines whether to start to detect the number of steps (Substep S402). For example, when the personal authentication of the user is successfully performed in Substep S401 described above and an operation indicating the intention to approve the detection of the number of steps is received from the user (Substep S402: Yes), the wearable device 100 or the mobile device 200 proceeds to Substep S403 in order to start to detect the number of steps. On the other hand, for example, when the personal authentication of the user fails in Substep S402 described above or when the operation indicating the intention to approve the detection of the number of steps is not received from the user, the wearable device 100 or the mobile device 200 repeats the processing of Substep S401 (Substep S402: No).

The wearable device 100 or the mobile device 200 sets data Dₛₜₑₚ about the number of steps stored to 0 and starts the detection of the number of steps (specifically, acquisition of the inertial data) (Substep S403).

The wearable device 100 or the mobile device 200 starts acquisition of the sensing data to acquire the features (Substep S404). Note that details of calculation of the features in the present embodiment will be described later.

The wearable device 100 or the mobile device 200 calculates (counts) the data Dₛₜₑₚ about the number of steps of the user on the basis of the change in the inertial data (the acceleration data, the angular velocity data, etc.) having ever been acquired (Substep S405). Note that, in the present embodiment, the number of steps of the user may be calculated by analyzing the inertial data, referring to the model obtained in advance by machine learning.

The wearable device 100 or the mobile device 200 determines whether there is no detection of the number of steps for a predetermined time period or more (Substep S406). When there is no detection of the number of steps for the predetermined time period or more (Substep S406: Yes), the wearable device 100 or the mobile device 200 proceeds to Substep S407. On the other hand, when the wearable device 100 or the mobile device 200 is not in the state there is no detection of the number of steps for the predetermined time period or more (i.e., the number of steps is detected), the process returns to Substep S405 (Substep S406: No).

The wearable device 100 or the mobile device 200 calculates the features, on the basis of the sensing data having ever been acquired, and calculates the reliability on the basis of the calculated features (Substep S407). Note that details of the features and the reliability in the present embodiment will be described later.

Note that, in the present embodiment, a timing interval for calculating the reliability and the time length of a sensing data acquisition period for calculating the features are preferably basically long, from the viewpoint of reliability. However, when the time length is too long, there is a high possibility of including many time slots during which the user is not walking. Therefore, in the present embodiment, it is preferable to set and adjust the time length in view of a sensing data acquisition state, a numerical value of the reliability, and a balance between a processing load, power consumption, and the like in the mobile device 200.

The wearable device 100 or the mobile device 200 determines whether the reliability calculated in Substep S407 described above is equal to or above the predetermined threshold (Substep S408). When the reliability is equal to or above the predetermined threshold (Substep S408: Yes), the wearable device 100 or the mobile device 200 proceeds to Substep S409. On the other hand, when the reliability is not equal to or above the predetermined threshold (Substep S408: No), the wearable device 100 or the mobile device 200 proceeds to Substep S410.

Note that, in the present embodiment, the predetermined threshold described above may be fixed to a preset value or may be determined or changed by the server 300, referring to a plurality of histories (the number of steps and the like) of a plurality of users or the results of the guarantee and management of the insurance company. With a configuration as described above, for example, even when the insurance premium is reduced for each user according to the result of the number of steps, the insurance company can achieve a benefit.

The wearable device 100 or the mobile device 200 outputs the number of steps having ever calculated to the server 300 (Substep S409).

The wearable device 100 or the mobile device 200 finishes the acquisition of the sensing data for acquiring the features, and the process returns to Substep S402 (Substep S410).

Note that, in the present embodiment, it is preferable to acquire the sensing data and calculate the number of steps, the features, and the like, only when the number of steps is detected, and such a configuration makes it possible to suppress the increase in processing load and power consumption in the wearable device 100 or the mobile device 200.

### <2.7 Features>

In the present embodiment, the reliability of the number of steps is calculated in order to confirm that the number of steps calculated as described above is not the number of steps falsified. Then, in the present embodiment, for calculation of the reliability, the features characterizing the plurality of pieces of sensing data are calculated, from the plurality of pieces of sensing data obtained by various sensors mounted in the wearable device 100, and the reliability is calculated using the calculated features. For example, in the present embodiment, the number of steps or walking distance estimated from the features is compared with the number of steps calculated using the inertial data, or the walking distance obtained by multiplying the number of steps by the stride registered in advance by the user, and when a difference in the number of steps or walking distance is small, it is determined that the number of steps is not the number of steps falsified. Then, in the present embodiment, the number of steps obtained by the inertial data can be regarded as the number of steps valid that can be reflected in the calculation of the insurance premium.

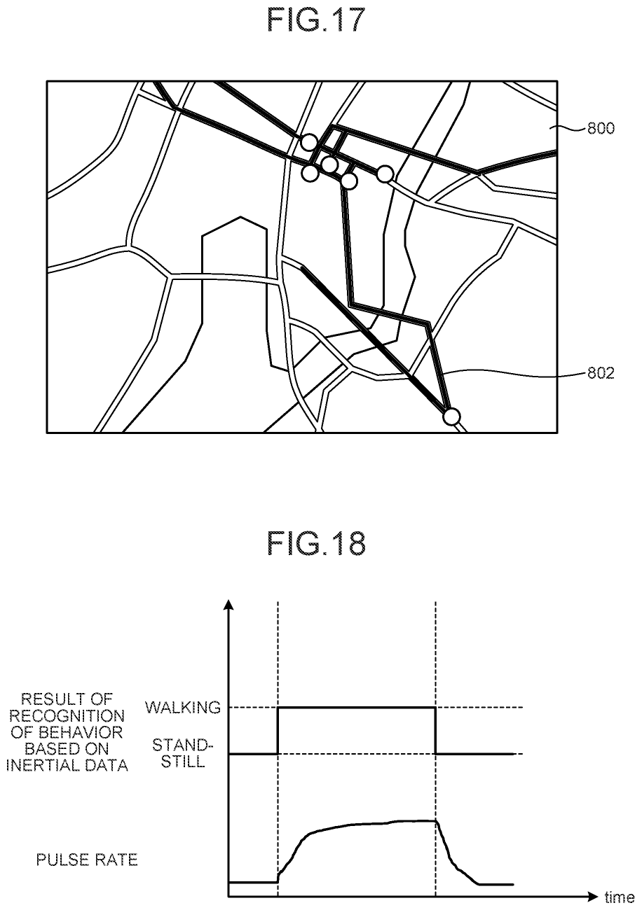

Specifically, in the present embodiment, the features can be calculated from the inertial data, the position data, the biological data, and the environment data included in the plurality of pieces of sensing data. Hereinafter, details of the pieces of data will be sequentially described with reference to FIGS. 17 to 21. Note that FIGS. 17 to 21 are each an illustrative diagram of an example of each feature in the present embodiment.

### (Inertial Data)

In the present embodiment, the inertial data is data that changes due to three-dimensional inertial motion (translational motion and rotational motion in three orthogonal axis directions) of the user, and specifically refers to the acceleration data, the angular velocity data, and the like. Specifically, in the present embodiment, as described above, it is possible to calculate the data Dₛₜₑₚ about the number of steps of the user on the basis of the change in the inertial data (the acceleration data, the angular velocity data, etc.). Furthermore, in the present embodiment, it is possible to calculate the walking distance as a feature by multiplying the calculated number of steps by the stride registered in advance by the user. Note that, in the present embodiment, when it is troublesome to measure the stride, for example, the height of the user may be multiplied by a predetermined coefficient (e.g., 0.45) so that the result is treated as a numerical value instead of the stride. Furthermore, in the present embodiment, the predetermined coefficient described above may be dynamically changed according to a result of the recognition of the user's behavior (e.g., walking, running, etc.) (e.g., since the stride changes between walking and running, the coefficient is increased for running relative to that for walking.).

Furthermore, in the present embodiment, the recognition of the user's behavior (running, walking, etc.) can be performed for one of the features, on the basis of the inertial data (the acceleration data, the angular velocity data, etc.), and a result of the recognition is referred to as the behavior recognition data. Here, the behavior recognition data means data indicating the movement or motion of the user, and specifically refers to data indicating the movement or motion, such as walking or running, of the user. Note that, in the present embodiment, at this time, the recognition of the behavior may be performed, referring to a model obtained in advance by machine learning using the inertial data of many users. Furthermore, in the present embodiment, the recognition of the behavior may be performed using a model obtained by machine learning using the inertial data obtained by the IMU 152 worn on a target user, and this configuration makes it possible to increase the accuracy in recognition of the behavior of a specific user.

Furthermore, in the present embodiment, not only the inertial data but also, for example, a schedule (wake-up time, clock-in time, quitting time, bedtime, etc.) input by the user in advance may be used for the recognition of the behavior. Alternatively, in the present embodiment, the recognition of the behavior may be performed using position data (sensing data) (e.g., home, company, school, station, etc.) obtained by the positioning sensor 154. In this way, the accuracy of the recognition of the behavior can be improved.

Furthermore, in the present embodiment, for example, in a case where the IMU 152 is mounted on both the wearable device 100 and the mobile device 200, recognition of the user's behavior may be performed by comparing the inertial data between both devices. More specifically, a time difference in peak of acceleration in a gravity direction between the wearable device 100 and the mobile device 200, which is within a predetermined time period, may be recognized as walking. Specifically, a periodic change in an arm swing direction in the acceleration data obtained by the wearable device 100 but no periodic change in the arm swing direction in the acceleration data obtained by the mobile device 200 is recognized as walking. Furthermore, satisfaction of these two conditions may be recognized as walking. In addition, in the present embodiment, in a case where, for example, it is determined, on the basis of the radio field intensity or the like, that short-distance communication can be performed between the wearable device 100 and the mobile device 200, that is, the wearable device 100 and the mobile device 200 are located within a predetermined distance (e.g., within 1 m), satisfaction of this condition and the above two conditions may be recognized as walking.

### (Position Data)

In the present embodiment, the position data is information indicating the position of the user in a global coordinate system or a relative coordinate system. Specifically, in the present embodiment, it is possible to acquire the position data of the user on the basis of the sensing data from the positioning sensor 154. For example, as illustrated in FIG. 17, a position of the user or a trajectory 802 thereof on a map 800 can be acquired as the position data on the basis of the sensing data from the positioning sensor 154. Furthermore, data about the walking distance of the user can be acquired as a feature, on the basis of a history of change in the position data. Note that when the user is located indoors, use of the GNSS signal causes a large error, and therefore, in the present embodiment, for example, it is preferable to combine the GNSS signal with pedestrian dead reckoning (indoor positioning technology) using Wi-Fi access points or the like. Furthermore, in the present embodiment, in order to improve the accuracy in distance, it is preferable to acquire the position data every short time (e.g., one minute) to calculate the distance.

### (Biological Data)

In the present embodiment, the biological data is information indicating the conditions of the body of the user, such as the pulse rate, heart rate, blood pressure, blood flow rate, respiration, skin temperature, perspiration, brain wave, myoelectric potential, and skin resistance level of the user. Specifically, in the present embodiment, it is possible to perform the recognition of the user's behavior, as the feature, from the biological data of the user based on the sensing data from the biological information sensor 156. For example, as illustrated in FIG. 18, the walking of the user can be recognized on the basis of a change in the pulse rate obtained from the biological information sensor 156. Specifically, the pulse rate (heart rate) is raised during walking relative to that during a standstill, and therefore, for example, an average value of the pulse rate (heart rate) for five minutes, which is significantly higher than the last value, may be recognized as walking. Alternatively, in the present embodiment, the pulse rate equal to or above a predetermined value may be recognized as walking or running. Furthermore, in the present embodiment, significant rising in in blood pressure, blood flow rate, respiration, skin temperature, and perspiration, in addition to the pulse rate (heart rate), or a significant change in brain wave, myoelectric potential, skin resistance level, and the like may be recognized as walking.

### (Environment Data)

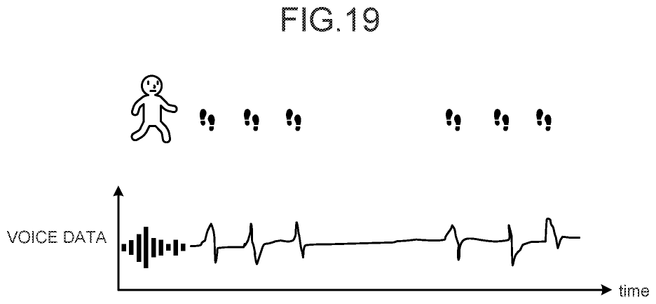

In the present embodiment, the environment data is, for example, information indicating the conditions of the environment around the user that are obtained as an image, sound, and radio wave (specifically, for example, change in radio field intensity). Specifically, in the present embodiment, it is possible to calculate the recognition of the behavior, the number of steps, the walking distance, and the like of the user, as the features, from the environment data based on the sensing data from the microphone 160, image sensor 158, and communication unit 180. For example, in the present embodiment, as illustrated in FIG. 19, it is possible to extract a characteristic change in sound of walking of the user on the basis of the change in sensing data of the microphone 160 and calculate the number of steps of the user can be calculated. Furthermore, in the present embodiment, for example, a magnitude of the correlation between the temporal change of the inertial data based on walking and the temporal change of the sound, which is equal to or above a predetermined value, may be recognized as walking.

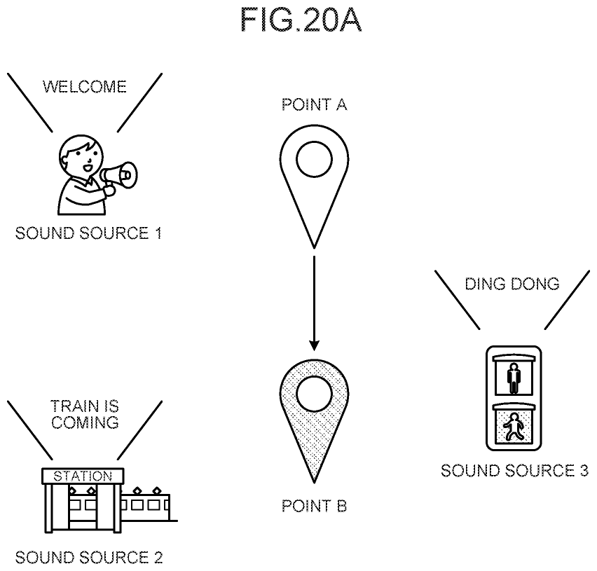

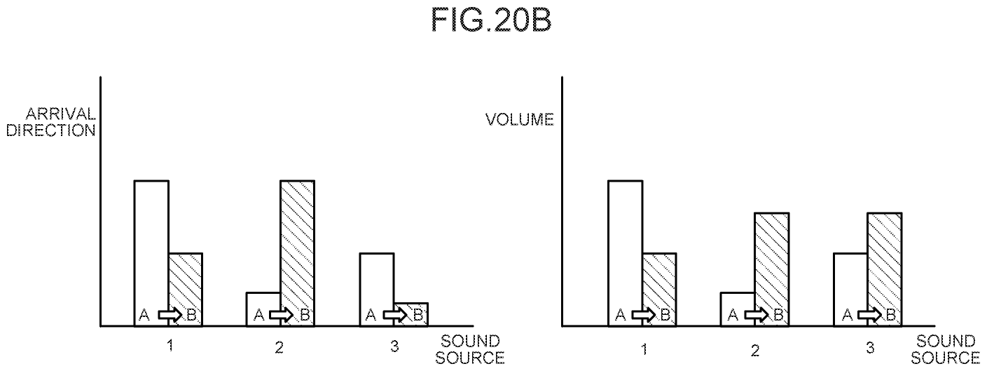

Furthermore, in the present embodiment, for example, as illustrated in FIGS. 20A and 20B, the movement of the user, that is, walking (running) may be detected as the feature, on the basis of changes in the arrival direction and volume of sound from each sound source included in the environmental sound around the user. Specifically, as illustrated in FIG. 20A, when the user moves (walks or runs) from a point A to a point B, the arrival direction and volume of the sound from each of sound sources 1 to 3 detected by the microphone 160 worn by the user should change. Therefore, in the present embodiment, sound is separated for each sound sources from the sounds detected by the microphone 160, and as illustrated in FIG. 20B, the changes in the arrival direction and volume of the sound from each of the sound sources 1 to 3 are analyzed as the features. Then, for example, the (temporal) changes that are found in the arrival direction and volume of sounds from three or more sound sources may be recognized as walking (running).

Furthermore, in the present embodiment, the movement of the user, that is, walking (running) may be detected as the feature, on the basis of a change in the intensity of a radio wave (e.g., WiFi (registered trademark), Bluetooth (registered trademark), etc.) detected by the communication unit 180. Specifically, when the user moves (walks or runs), the intensity of the radio wave from each access point, detected by the communication unit 180 of the wearable device 100 worn by the user should change. Therefore, in the present embodiment, when the radio field intensity may be detected for each piece of identification information (e.g., SSID) of the access point, the intensity of the radio wave having the same identification information that changes even after a certain period of time may be recognized as walking (running). Furthermore, in the present embodiment, an attenuation rate of the radio field intensity can be applied to a predetermined formula to calculate the movement distance of the user, and therefore, it is also possible to calculate a movement distance of the user per time, that is, a speed. Then, the calculated speed is compared with a predetermined threshold, and it is possible to determine whether the user is walking or running. Furthermore, dividing the distance by the stride of the user (the stride may be changed between walking and running) makes it possible to calculate the number of steps of the user.

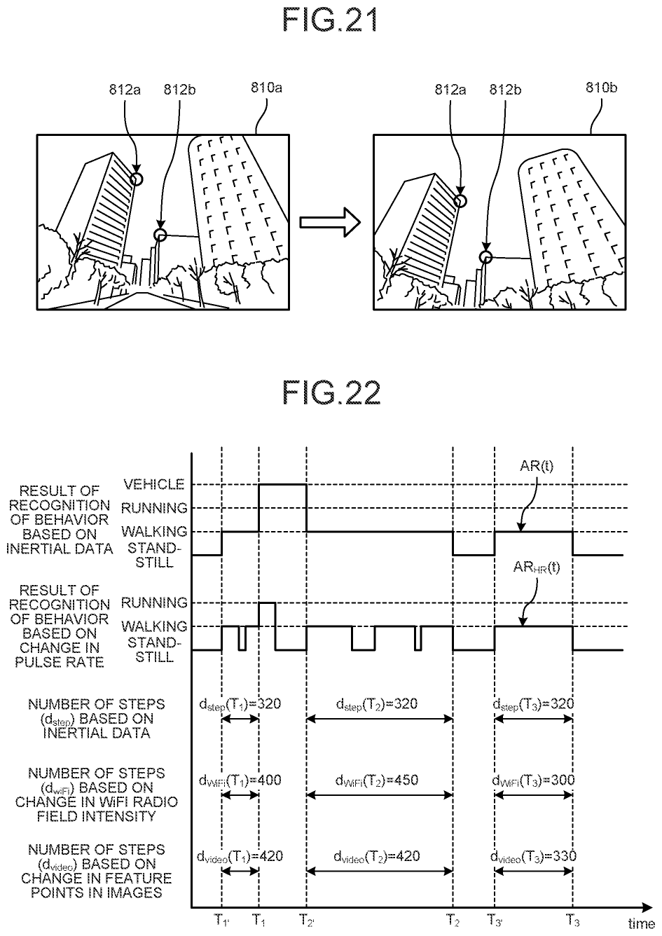

Furthermore, in the present embodiment, the user's behavior or walking distance may be detected as the feature, on the basis of the change of an image acquired by the image sensor 158. Specifically, as illustrated in FIG. 21, when the user moves (walks or runs), the image around the user acquired by the image sensor 158 worn by the user should change. Therefore, feature points (e.g., edges 812a and 812b of buildings) of subjects are extracted from each of images 810a and 810b acquired continuously in a short time, and on the basis of a change in a relative positional relationship (coordinates) between the same feature points or a change in direction between the images 810a and 810b, the behavior (walking, running, etc.) of the user can be recognized or the speed or distance can be calculated. Furthermore, as described above, dividing the distance by the stride of the user (the stride may be changed between walking and running) makes it possible to calculate the number of steps of the user.

Note that the present embodiment is not limited to the sensing data acquired by the various sensors described above and the features obtained from the sensing data, and may be, but not particularly limited, sensing data or the features obtained from another sensor.

### <2.8 Calculation of Reliability>

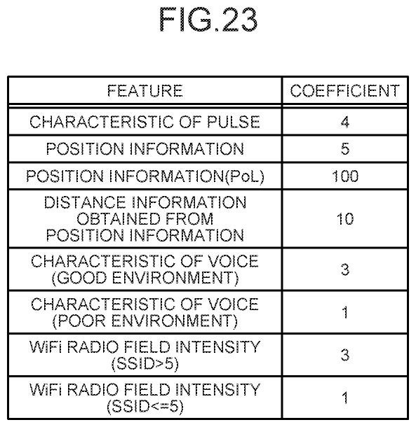

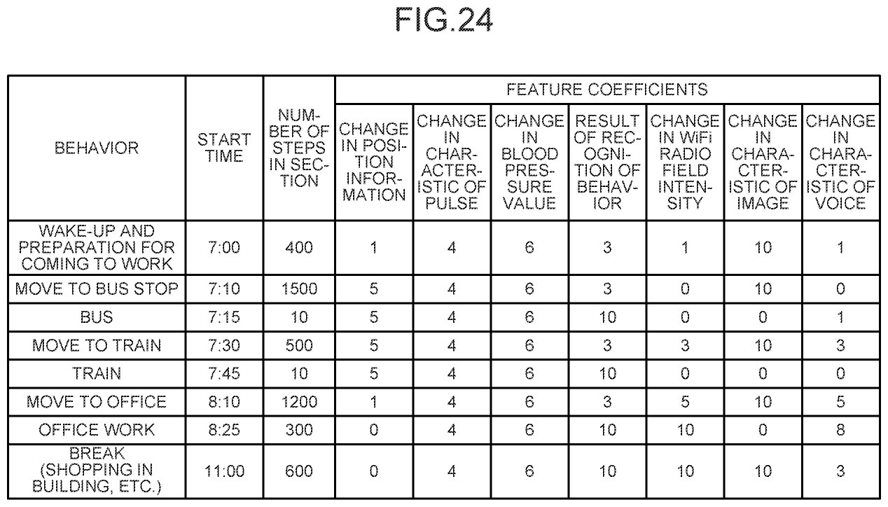

Next, details of calculation of the reliability according to the present embodiment will be described with reference to FIGS. 22 to 24. FIG. 22 is an illustrative diagram illustrating the reliability in the present embodiment, and FIGS. 23 and 24 are each a table illustrating exemplary coefficients in the present embodiment.

It is considered that the reliability of the recognition of the behavior (walking and running) and the reliability of the distance or the number of steps are different depending on the type of the feature. Therefore, in the present embodiment, upon calculating the reliability, weighting (coefficient) in obtaining the reliability is changed for each type of feature while using a plurality of features.

Specifically, in the present embodiment, as illustrated in FIG. 22, when the recognition of the behavior and the number of steps are obtained, a reliability rᵢ can be obtained by, for example, the following mathematical formula (1). In this configuration, the recognition of the behavior is obtained on the basis of the inertial data and the change in the pulse rate, and the number of steps is obtained on the basis of the inertial data, the change in the radio field intensity, and the change in the feature in the images.

$$\\ \begin{matrix}{r}_{i} = \frac{\frac{{\sum }_{τ = {T}_{{i}^{{}_{} ′}}}^{{T}_{i}} \{ AR ( τ ) - {AR}_{HR} ( τ ) \}}{i - {i}^{{}_{} ′} + 1} \times {Coeff}_{HR} + \frac{{d}_{WiFi} ( {T}_{i} ) - {d}_{step} ( {T}_{i} )}{{d}_{step} ( {T}_{i} )} \times {coeff}_{WiFi} + \frac{{d}_{video} ( {T}_{i} ) - {d}_{step} ( {T}_{i} )}{{d}_{step} ( {T}_{i} )} \times {coeff}_{video}}{( {coeff}_{HR} + {coeff}_{WiFi} + {coeff}_{video} )} & ( 1 )\end{matrix}$$

Then, in the present embodiment, for a coefficient Coeff_{HR} (coefficient for the number of steps based on the change in the pulse rate), a coefficient Coeff_{WiFi} (coefficient for the number of steps based on the change in the radio field intensity), and a coefficient Coeff_{video} (coefficient for the number of steps based on the change in the feature points in the images) of mathematical formula (1), values determined in advance according to the properties of the features can be used. More specifically, for the characteristics related to the distance and the number of steps, a difference from each of the distance and the number of steps that are obtained from the inertial data is calculated, a mean value (variance value) or a normalized value of the differences is multiplied by each coefficient (coefficient ratio) and added together, and thus, the reliability rᵢ can be obtained.

Note that, in the present embodiment, the calculation formula of the reliability rᵢ is not limited to the above mathematical formula (1), and is not particularly limited as long as a formula in which the respective features are weighted (coefficient) in calculation.

Furthermore, in the present embodiment, as illustrated in FIG. 23, the coefficient for each feature may be dynamically changed according to a feature acquisition state. In the present embodiment, for example, the coefficient related to the feature obtained from the position data of the user by using the GNSS signal may be changed depending on whether the PoL technology is used. Furthermore, in the present embodiment, for example, the coefficient related to the feature obtained from the voice data may be changed according to the voice acquisition state (e.g., a noise ratio or the like). Furthermore, in the present embodiment, for example, the coefficient related to a characteristic obtained from the environment data by acquisition of a radio wave may be changed according to the number of radio wave sources (the number of access points) of radio waves acquired.

Furthermore, in the present embodiment, as illustrated in FIG. 24, the coefficient for each feature may be dynamically changed according to the behavior of the user or the position of the user. In the present embodiment, for example, there is a high possibility that the number of access points is small at the user's home or near the user's home, and therefore, a small coefficient is preferably set for the feature based on the change in the radio field intensity. Meanwhile, in an indoor space such as a company, higher accuracy is expected due to a large number of access points, and therefore, a large coefficient is preferably set for the feature based on the change in the radio field intensity. Furthermore, in the present embodiment, for example, when the user is walking outdoors, a large coefficient is preferably set for the feature based on the change in the feature points in the images. Furthermore, in the present embodiment, for example, when the user is on a bus, the number of steps has less change, and therefore, the coefficient for the feature of the recognition of the behavior is preferably increased.

Note that, in the present embodiment, the values of the coefficients are not limited to the values illustrated in FIGS. 23 and 24, and can be appropriately selected.

### 3. CONCLUSION

As described above, according to the embodiments of the present disclosure, it is possible to prevent the falsification of the number of steps or the like, and therefore, the insurance premium is calculated by using the fair steps or the like, providing the sense of satisfaction with the insurance premium. Therefore, the health of the insured person is enhanced, and an increase in the number of insurance subscribers is promising. Note that, in the description of the above embodiments, an exemplary application to the prevention of falsification of the number of steps has been described, but the present embodiment is not limited thereto, and can also be applied to the prevention of falsification of the movement distance of the user.

Note that, in the above description, although it has been described that the number of steps and the reliability are calculated mainly by the mobile device 200 and the insurance premium is calculated by the server 300, the embodiments of the present disclosure are not limited to such a form. In the embodiments of the present disclosure, for example, in one or both of the wearable device 100 and the mobile device 200, the processing from the calculation of the number of steps and the reliability to the calculation of the insurance premium may be performed, and whole or part of the processing may be performed by a large number of cloud information processing devices.

Note that, as described above, the embodiments of the present disclosure are not limited to application to the information processing for acquiring the number of steps for the health promotion insurance. The embodiments of the present disclosure may be applied to, for example, a service of giving discount points (incentive) that can be used for shopping or the like instead of cash to the user depending on the number of steps, or a service of giving health advice to the user depending on the number of steps.

### 4. HARDWARE CONFIGURATION

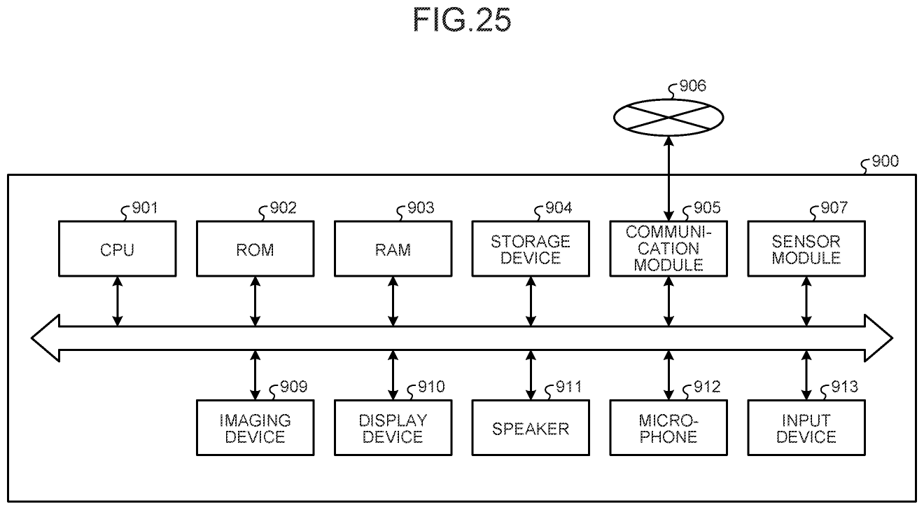

FIG. 25 is a block diagram illustrating an example of a schematic functional configuration of a smartphone 900, and for example, the smartphone 900 can be the mobile device 200 described above. Therefore, a configuration example of the smartphone 900 as the mobile device 200 to which an embodiment of the present disclosure is applied will be described with reference to FIG. 25.

As illustrated in FIG. 25, the smartphone 900 includes a central processing unit (CPU) 901, a read only memory (ROM) 902, and a random access memory (RAM) 903. In addition, the smartphone 900 includes a storage device 904, a communication module 905, and a sensor module 907. Furthermore, the smartphone 900 includes an imaging device 909, a display device 910, a speaker 911, a microphone 912, an input device 913, and a bus 914. Furthermore, the smartphone 900 may include a processing circuit such as a digital signal processor (DSP) instead of or in addition to the CPU 901.

The CPU 901 functions as an arithmetic processing unit and a control device, and controls all or some of operations in the smartphone 900, according to various programs recorded in the ROM 902, the RAM 903, the storage device 904, or the like. The ROM 902 stores programs, calculation parameters, and the like used by the CPU 901. The RAM 903 primarily stores the programs used in execution of the CPU 901 or parameters or the like that change appropriately in the execution of the programs. The CPU 901, the ROM 902, and the RAM 903 are connected to each other by the bus 914. Furthermore, the storage device 904 is a data storage device configured as an example of a storage unit of the smartphone 900. The storage device 904 includes, for example, a magnetic storage device such as a hard disk drive (HDD), a semiconductor storage device, an optical storage device, or the like. The storage device 904 stores the programs and various data executed by the CPU 901, various data acquired from outside, and the like.

The communication module 905 is a communication interface that includes, for example, a communication device or the like for connection to a communication network 906. The communication module 905 can be, for example, a communication card or the like for a wired or wireless local area network (LAN), Bluetooth (registered trademark), or a wireless USB (WUSB). Furthermore, the communication module 905 may be a router for optical communication, a router for an asymmetric digital subscriber line (ADSL), a modem for various communications, or the like. The communication module 905 transmits and receives signals and the like to and from the Internet or other communication devices by using a predetermined protocol such as Transmission Control Protocol (TCP)/Internet Protocol (IP). Furthermore, the communication network 906 connected to the communication module 905 is a network connected in a wired or wireless manner, and is, for example, the Internet, a home LAN, infrared communication, satellite communication, or the like.

The sensor module 907 includes various sensors, such as a motion sensor (e.g., acceleration sensor, gyroscope sensor, geomagnetic sensor, etc.), a biological information sensor (e.g., pulse sensor, blood pressure sensor, fingerprint sensor, etc.), and a position sensor (e.g., global navigation satellite system (GNSS) receiver, and the like).

The imaging device 909 is provided on a surface of the smartphone 900 to image a target or the like positioned on the front or back side of the smartphone 900. Specifically, the imaging device 909 is configured to include an imaging element (not illustrated) such as a complementary MOS (CMOS) image sensor, and a signal processing circuit (not illustrated) that performs imaging signal processing on a signal obtained by photoelectric conversion by the imaging element. Furthermore, the imaging device 909 can further include an optical system mechanism (not illustrated) that includes an imaging lens, a zoom lens, a focusing lens, and the like, and a drive system mechanism (not illustrated) that controls the operations of the optical system mechanism. Then, the imaging element collects incident light from the target, as an optical image, and the signal processing circuit photoelectrically converts the formed optical image in units of pixels, reads a signal of each pixel as an imaging signal, and performs image processing to acquire a captured image.

The display device 910 is provided on a surface of the smartphone 900 and can be a display device such as a liquid crystal display (LCD) or organic electro luminescence (EL) display. The display device 910 is configured to display an operation screen, the captured image acquired by the imaging device 909 described above, and the like.

The speaker 911 is configured to output, for example, a voice call, voice accompanying image content displayed by the display device 910 described above, and the like, to the user.

The microphone 912 is configured to collect, for example, a voice call of the user, a voice including a command for activating a function of the smartphone 900, and sound in a surrounding environment of the smartphone 900.

The input device 913 is a device such as a button, touch screen, or mouse that is operated by the user. The input device 913 includes an input control circuit that generates an input signal on the basis of information input by the user and that outputs the input signal to the CPU 901. The user can operate the input device 913 to input various data to the smartphone 900 or give an instruction for processing operation.

An exemplary hardware configuration of the smartphone 900 has been described above. Note that the hardware configuration of the smartphone 900 is not limited to the configuration illustrated in FIG. 25. Specifically, each of the component elements described above may include a general-purpose member, or may include hardware specialized for the function of each component element. This configuration can be appropriately changed according to the technical level when the present embodiment is implemented.

Furthermore, the smartphone 900 according to the present embodiment may be applied to a system including a plurality of devices on the premise of connection to a network (or communication between the devices), such as cloud computing. In other words, the mobile device 200 according to the present embodiment described above can also be implemented, for example, as the information processing system 10 that performs the processing related to the information processing method according to the present embodiment by using the plurality of devices.

### 5. SUPPLEMENTARY NOTES

Preferred embodiments of the present disclosure have been described above in detail with reference to the accompanying drawings, but the technical scope of the present disclosure is not limited to these examples. A person skilled in the art may obviously find various alternations or modifications within the technical concept described in claims, and it should be understood that the alternations and modifications will naturally come under the technical scope of the present disclosure.

Note that the embodiments of the present disclosure described above can include, for example, a program for causing a computer to function as the information processing device according to the present embodiment, and a non-transitory tangible medium on which the program is recorded. In addition, the program may be distributed via a communication line (including wireless communication) such as the Internet.

Furthermore, the respective steps in the processing of each embodiment described above may not necessarily be processed in the order having been described. For example, the order of the processing may be appropriately changed between the respective steps. In addition, the processing of the respective steps may be performed partially in parallel or individually, instead of being performed in chronological order. Furthermore, the processing method of each step may not be necessarily performed according to the described method, and may be performed, for example, by another method by using another functional unit.

Furthermore, the effects descried herein are merely illustrative or exemplified effects, and are not limitative. In other words, with or in place of the above effects, the technology according to the present disclosure can provide other effects that are apparent to those skilled in the art from the description herein.

Note that the present technology can also have the following configurations.

(1) An information processing device comprising:
a sensing data acquisition unit that acquires a plurality of pieces of sensing data from a device worn by a user or carried by the user;
a calculation unit that calculates the number of steps or a movement distance of the user based on inertial data included in the plurality of pieces of sensing data;
a reliability calculation unit that calculates reliability based on a feature of each of position data, biological data, and environment data about the user obtained from the plurality of pieces of sensing data;
a determination unit that determines whether to accept the number of steps or movement distance calculated, based on the reliability calculated; and
an output unit that outputs data of the number of steps or movement distance received.
   (2) The information processing device according to (1), wherein the reliability calculation unit calculates the reliability based on a feature of behavior recognition data about the user.
   (3) The information processing device according to (2), further comprising
a feature calculation unit that calculates a feature from each of the inertial data, the position data, the biological data, and the environment data included in the plurality of pieces of sensing data.
   (4) The information processing device according to (3), wherein the feature calculation unit calculates the feature by performing statistical processing on at least one of the plurality of pieces of sensing data.
   (5) The information processing device according to (3), wherein the feature calculation unit calculates the feature by referring to a model obtained in advance by machine learning.
   (6) The information processing device according to (3), wherein
the feature calculation unit
calculates first distance data, from at least one of the plurality of pieces of sensing data, and
calculates a difference between the first distance data and second distance data obtained based on the number of steps, as the feature.
   (7) The information processing device according to (3), wherein
the feature calculation unit
recognizes a user's behavior, based on at least one of the plurality of pieces of sensing data, and calculates the feature from a result of the recognition.
   (8) The information processing device according to (7), wherein
the sensing data acquisition unit acquires the plurality of pieces of sensing data from a wearable device worn by the user and a mobile device carried by the user, and
the feature calculation unit compares the same type of sensing data between the respective devices to recognize the user's behavior.
   (9) The information processing device according to any one of (2) to (8), wherein the reliability calculation unit calculates the reliability by weighting each feature with a predetermined coefficient given to each feature.
   (10) The information processing device according to (9), wherein the reliability calculation unit dynamically changes the predetermined coefficient according to at least one of a position of the user, the behavior recognition data, and a change in position of the user.
   (11) The information processing device according to any one of (1) to (10), wherein the determination unit compares the reliability with a predetermined threshold to determine whether to accept the number of steps or movement distance calculated.
   (12) The information processing device according to (11), wherein the determination unit dynamically changes the predetermined threshold.
   (13) The information processing device according to any one of (1) to (12), wherein the inertial data included in the plurality of pieces of sensing data is acquired from an acceleration sensor, an angular velocity sensor, or a geomagnetic sensor worn or carried by the user.
   (14) The information processing device according to any one of (1) to (13), wherein the environment data is generated from sensing data acquired from a voice sensor, an image sensor, or a radio wave sensor worn by the user or carried by the user.
   (15) The information processing device according to any one of (1) to (14), wherein
the output unit outputs data of the number of steps or movement distance received, to a server that calculates an incentive to the user, and
the information processing device further includes a presentation unit that presents, to the user, the incentive calculated by the server based on the data of the number of steps or movement distance.
   (16) The information processing device according to (15), wherein the incentive is a discount amount of an insurance premium for the user.
   (17) The information processing device according to any one of (1) to (16), further comprising an authentication unit that authenticates the user.
   (18) An information processing system comprising:
a server that calculates an incentive to a user; and
an information processing device that is worn by the user or carried by the user,
wherein the information processing device includes:
a sensing data acquisition unit that acquires a plurality of pieces of sensing data from a device worn by the user or carried by the user;
a calculation unit that calculates the number of steps or a movement distance of the user based on inertial data included in the plurality of pieces of sensing data;
a reliability calculation unit that calculates reliability based on a feature of each of position data, biological data, and environment data about the user obtained from the plurality of pieces of sensing data;
a determination unit that determines whether to accept the number of steps or movement distance calculated, based on the reliability calculated;
an output unit that outputs, to the server, data of the number of steps or movement distance received; and
a presentation unit that presents, to the user, the incentive calculated by the server based on the data of the number of steps or movement distance.
   (19) An information processing method comprising:
by an information processing device,
acquiring a plurality of pieces of sensing data from a device worn by a user or carried by the user;
calculating the number of steps or a movement distance of the user based on inertial data included in the plurality of pieces of sensing data;
calculating reliability based on a feature of each of position data, biological data, and environment data about the user obtained from the plurality of pieces of sensing data;
determining whether to accept the number of steps or movement distance calculated, based on the reliability calculated; and
outputting data of the number of steps or movement distance received.
   (20) A program causing
a computer to perform:
a function of acquiring a plurality of pieces of sensing data from a device worn by a user or carried by the user;
a function of calculating the number of steps or a movement distance of the user based on inertial data included in the plurality of pieces of sensing data;
a function of calculating reliability based on a feature of each of position data, biological data, and environment data about the user obtained from the plurality of pieces of sensing data;
a function of determining whether to accept the number of steps or movement distance calculated, based on the reliability calculated; and
a function of outputting data of the number of steps or movement distance received.

### REFERENCE SIGNS LIST

10 INFORMATION PROCESSING SYSTEM
100 WEARABLE DEVICE
110, 210, 310 INPUT UNIT
120, 242 AUTHENTICATION INFORMATION ACQUISITION UNIT
130, 230, 330 DISPLAY UNIT
140 CONTROL UNIT
150 SENSOR UNIT
152 IMU
154 POSITIONING SENSOR
156 BIOLOGICAL INFORMATION SENSOR
158 IMAGE SENSOR
160 MICROPHONE
170, 270, 370 STORAGE UNIT
180, 280, 380 COMMUNICATION UNIT
200 MOBILE DEVICE
240, 340 PROCESSING UNIT
244 AUTHENTICATION UNIT
246 SENSING DATA ACQUISITION UNIT
248 STEP CALCULATION UNIT
250 FEATURE CALCULATION UNIT
252 RELIABILITY CALCULATION UNIT
254 DETERMINATION UNIT
256, 356 OUTPUT UNIT
260 INSURANCE PREMIUM INFORMATION ACQUISITION UNIT
300 SERVER
342 STEP INFORMATION ACQUISITION UNIT
344 INSURANCE PREMIUM CALCULATION UNIT
346 THRESHOLD CALCULATION UNIT
400 NETWORK
800 MAP
802 TRAJECTORY
810a, 810b IMAGE
812a, 812b EDGE
900 SMARTPHONE
901 CPU
902 ROM
903 RAM
904 STORAGE DEVICE
905 COMMUNICATION MODULE
906 COMMUNICATION NETWORK
907 SENSOR MODULE
909 IMAGING DEVICE
910 DISPLAY DEVICE
911 SPEAKER
912 MICROPHONE
913 INPUT DEVICE
914 BUS

1. An information processing device comprising:
a sensing data acquisition unit that acquires a plurality of pieces of sensing data from a device worn by a user or carried by the user;
a calculation unit that calculates the number of steps or a movement distance of the user based on inertial data included in the plurality of pieces of sensing data;
a reliability calculation unit that calculates reliability based on a feature of each of position data, biological data, and environment data about the user obtained from the plurality of pieces of sensing data;
a determination unit that determines whether to accept the number of steps or movement distance calculated, based on the reliability calculated; and
an output unit that outputs data of the number of steps or movement distance received.

2. The information processing device according to claim 1, wherein the reliability calculation unit calculates the reliability based on a feature of behavior recognition data about the user.

3. The information processing device according to claim 2, further comprising
a feature calculation unit that calculates a feature from each of the inertial data, the position data, the biological data, and the environment data included in the plurality of pieces of sensing data.

4. The information processing device according to claim 3, wherein the feature calculation unit calculates the feature by performing statistical processing on at least one of the plurality of pieces of sensing data.

5. The information processing device according to claim 3, wherein the feature calculation unit calculates the feature by referring to a model obtained in advance by machine learning.

6. The information processing device according to claim 3, wherein
the feature calculation unit
calculates first distance data, from at least one of the plurality of pieces of sensing data, and
calculates a difference between the first distance data and second distance data obtained based on the number of steps, as the feature.

7. The information processing device according to claim 3, wherein
the feature calculation unit
recognizes a user's behavior, based on at least one of the plurality of pieces of sensing data, and calculates the feature from a result of the recognition.

8. The information processing device according to claim 7, wherein
the sensing data acquisition unit acquires the plurality of pieces of sensing data from a wearable device worn by the user and a mobile device carried by the user, and
the feature calculation unit compares the same type of sensing data between the respective devices to recognize the user's behavior.

9. The information processing device according to claim 2, wherein the reliability calculation unit calculates the reliability by weighting each feature with a predetermined coefficient given to each feature.

10. The information processing device according to claim 9, wherein the reliability calculation unit dynamically changes the predetermined coefficient according to at least one of a position of the user, the behavior recognition data, and a change in position of the user.

11. The information processing device according to claim 1, wherein the determination unit compares the reliability with a predetermined threshold to determine whether to accept the number of steps or movement distance calculated.

12. The information processing device according to claim 11, wherein the determination unit dynamically changes the predetermined threshold.

13. The information processing device according to claim 1, wherein the inertial data included in the plurality of pieces of sensing data is acquired from an acceleration sensor, an angular velocity sensor, or a geomagnetic sensor worn or carried by the user.

14. The information processing device according to claim 1, wherein the environment data is generated from sensing data acquired from a voice sensor, an image sensor, or a radio wave sensor worn by the user or carried by the user.

15. The information processing device according to claim 1, wherein
the output unit outputs data of the number of steps or movement distance received, to a server that calculates an incentive to the user, and
the information processing device further includes a presentation unit that presents, to the user, the incentive calculated by the server based on the data of the number of steps or movement distance.

16. The information processing device according to claim 15, wherein the incentive is a discount amount of an insurance premium for the user.

17. The information processing device according to claim 1, further comprising an authentication unit that authenticates the user.

18. An information processing system comprising:
a server that calculates an incentive to a user; and
an information processing device that is worn by the user or carried by the user,
wherein the information processing device includes:
a sensing data acquisition unit that acquires a plurality of pieces of sensing data from a device worn by the user or carried by the user;
a calculation unit that calculates the number of steps or a movement distance of the user based on inertial data included in the plurality of pieces of sensing data;
a reliability calculation unit that calculates reliability based on a feature of each of position data, biological data, and environment data about the user obtained from the plurality of pieces of sensing data;
a determination unit that determines whether to accept the number of steps or movement distance calculated, based on the reliability calculated;
an output unit that outputs, to the server, data of the number of steps or movement distance received; and
a presentation unit that presents, to the user, the incentive calculated by the server based on the data of the number of steps or movement distance.

19. An information processing method comprising:
by an information processing device,
acquiring a plurality of pieces of sensing data from a device worn by a user or carried by the user;
calculating the number of steps or a movement distance of the user based on inertial data included in the plurality of pieces of sensing data;
calculating reliability based on a feature of each of position data, biological data, and environment data about the user obtained from the plurality of pieces of sensing data;
determining whether to accept the number of steps or movement distance calculated, based on the reliability calculated; and
outputting data of the number of steps or movement distance received.

20. A program causing
a computer to perform:
a function of acquiring a plurality of pieces of sensing data from a device worn by a user or carried by the user;
a function of calculating the number of steps or a movement distance of the user based on inertial data included in the plurality of pieces of sensing data;
a function of calculating reliability based on a feature of each of position data, biological data, and environment data about the user obtained from the plurality of pieces of sensing data;
a function of determining whether to accept the number of steps or movement distance calculated, based on the reliability calculated; and
a function of outputting data of the number of steps or movement distance received.
